(19) United States
(12) Patent Application Publication
(10) Pub. No.: US 2026/0047842 A1
(43) Pub. Date: Feb. 19, 2026

(54) SUTURING SYSTEM WITH PRESSURE FOOT

(71) Applicant: Bastion Labworks LLC, West Palm Beach, FL (US)

(72) Inventors: Frank THISSEN, Manahawkin, NJ (US); Juan E. VEGA-NEVAREZ, Jersey City, NJ (US)

(73) Assignee: Bastion Labworks LLC, West Palm Beach, FL (US)

(21) Appl. No.: 19/370,676

(22) Filed: Oct. 27, 2025

Related U.S. Application Data
(63) Continuation-in-part of application No. 19/224,654, filed on May 30, 2025.
(60) Provisional application No. 63/654,344, filed on May 31, 2024.
(60) Provisional application No. 63/721,358, filed on Nov. 15, 2024.

(51) Int. Cl. A61B 17/06; A61B 17/04; A61B 17/10

(52) U.S. Cl. CPC A61B 17/06166; A61B 17/06004; A61B 17/06061; A61B 17/105; A61B 2017/0496; A61B 2017/06042; A61B 2017/06176

(57) ABSTRACT

A suturing system includes a suturing device for use with a suture segment having a first suture segment barb and a second suture segment barb. The suturing device includes a first shaft terminating at a tip and having a first barb catch above the tip and a second shaft terminating at a second tip and having a second barb catch above the second tip.

### CROSS-REFERENCE TO RELATED PATENT APPLICATIONS

The present application is a continuation-in-part of co-pending U.S. patent application Ser. No. 19/224,654 (Atty. Dkt. No. B170-107-US), filed May 30, 2025, by Thissen et al. and entitled SUTURING SYSTEM WITH PRESSURE FOOT, which claims the benefit of U.S. Provisional Patent Application Ser. No. 63/721,358 (Atty. Dkt. No. B170-103-USP), filed Nov. 15, 2024, by Thissen et al. and entitled SUTURING SYSTEM and U.S. Provisional Patent Application Ser. No. 63/654,344 (Atty. Dkt. No. B170-102-USP), filed May 31, 2024, by Thissen et al. and entitled SUTURING SYSTEM, the full disclosures of which are hereby incorporated by reference.

### BACKGROUND

Suturing is used to join tissue to facilitate healing.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### BRIEF DESCRIPTION OF THE DRAWINGS

FIG. 1 is a sectional view schematically illustrating portions of an example suturing system.

FIG. 2 is a bottom view of the example suturing system of FIG. 1.

FIG. 3 is a sectional view of the example suturing system of FIG. 1 upon removal of an example guard and prior to implantation of suture segments.

FIG. 4 is a sectional view of the example suturing system of FIG. 1 following implantation of suture segments and following retraction of suture segment implanting prongs.

FIG. 5 is a sectional view schematically illustrating portions of an example suturing system and an unloaded state ready for loading.

FIG. 6 is a sectional view of the example suturing system of FIG. 5 and a ready and loaded state.

FIG. 7 is a bottom view of the suturing system of FIG. 6.

FIG. 8 is a sectional view of the example suturing system of FIG. 5 during implantation of suture segments.

FIG. 9 is a sectional view of the example suturing system of FIG. 5 following implantation of the suture segments and while an example pressure foot is pressing the implanted suture segments as the implanting prongs are being retracted.

FIG. 10 is a sectional view of the example suturing system of FIG. 5 following implantation of the suture segments and following retraction of the implanting prongs and the example pressure foot.

FIG. 11 is a perspective view of an example prong a carriage having a stack of spaced sections.

FIG. 12 is a perspective view of an example section for use in the example prong carriage of FIG. 11.

FIG. 13 is a perspective view of an example section for use in the example prong carriage of FIG. 11.

FIG. 14 the top perspective view of an example suturing system.

FIG. 15 is a bottom perspective view of the example suturing system of FIG. 42.

FIG. 16 is an exploded perspective view of the suturing system of FIG. 42.

FIG. 17 is a perspective view of an example pressure foot of the example suturing system of FIG. 16.

FIG. 18 is a perspective view of an example needle holder of the example suturing system of FIG. 16.

FIG. 19 is a perspective view of an example needle of the example suturing system of FIG. 16.

FIG. 20 is a sectional view of the example suturing system of FIG. 42.

FIG. 21 is a cross-sectional view of the example suturing system of FIG. 42.

FIG. 22 is a perspective view illustrating the example suturing system of FIG. 42 inverted during loading of suture segments.

FIG. 23 is a perspective view of the example suturing system of FIG. 42 with an example retainer securing the suture segments and an example optional shim for controlling implantation depth.

FIG. 24 is a top perspective view of the suturing system of FIG. 23.

FIG. 25 is a cross-sectional view of the suturing system of FIG. 23.

FIG. 26 is a bottom perspective view of the suturing system of FIG. 23 with another example retainer securing the needle holder in an extended position.

FIG. 27 sectional view of the suturing system of FIG. 26.

FIG. 28 is a perspective view of an example suturing system of FIG. 14 during implantation of suture segments into tissue.

FIG. 29 is a sectional view of the suturing system of FIG. 28.

FIG. 30 is a bottom perspective view of the suturing system of FIG. 28 with needles at a first implantation depth.

FIG. 31 is a bottom perspective view of the suturing system of FIG. 28 with an inserted shim and with needles at a second shallower implantation depth.

FIG. 32 is a perspective view of the suturing system of FIG. 28 following withdrawal of needles from the underlying tissue.

FIG. 33 is a cross-sectional view of the suturing system of FIG. 32.

FIG. 34 is a sectional view of the suturing system of FIG. 32.

FIG. 35 is a bottom perspective view of the suturing system of FIG. 42 illustrating needles with a first throw.

FIG. 36 is a bottom perspective view of the suturing system of FIG. 42 illustrating needles providing a shortened suture length.

FIG. 37 is a perspective view of an example suturing system.

FIG. 38 is a sectional view of the suturing system of FIG. 37.

FIG. 39 is a top view of an example suturing system.

FIG. 40 is a bottom view of the example suturing system of FIG. 39.

FIG. 41 is a perspective view of an example suturing system withdraws in an open state.

FIG. 42 is a perspective view of the suturing system of FIG. 41 with the jaws in a closed state.

FIG. 43 is a perspective view schematically illustrating portions of an example suturing system with an example staple loaded with an example suture segment.

FIG. 44 is a side view of an example prong for the example staple of the example suturing system of FIG. 43.

FIG. 45 is a second side view of the example prong of FIG. 44.

FIG. 46 is a side view of an example end portion of an example suture segment.

FIG. 47 is a sectional view of the example suture segment of FIG. 46 caught or engaged by the example prong of FIGS. 44 and 45 during implantation of the example suture segment.

FIG. 48 is a perspective view of an example staple having example prongs with example barb catches.

FIG. 49 is a perspective view of an example staple having example prongs with example barb catches.

FIG. 50 is a perspective view of an example staple having example prongs with example barb catches.

FIG. 51 is a perspective view of an example needle providing an example prong with an example barb catch.

FIG. 52 is a side view of an example prong of an example staple or needle having an example barb catch engage with an example barb of an example suture segment during implantation.

FIG. 53 is a front view of an example prong of an example staple or needle having an example barb catch for implanting a suture segment.

FIG. 54 is a side view of the example prong of FIG. 53 engaged with a barb of an example suture segment during implantation.

FIG. 55 is a side view of an example prong of a staple or needle engage with a barb of an example suture segment during implantation.

FIG. 56 is a front view of an example prong of an example staple or needle engage with an example barb of an example suture segment during implantation.

FIG. 57 is a side view of the example prong of FIG. 56 engaging the example suture segment during implantation.

FIG. 58 is an enlarged view of a portion of the prong of FIG. 57 engaging the example barb of the example suture segment during implantation.

FIG. 59 is a top view of an example one piece suture segment.

FIG. 60 is a perspective view of an example suturing system including an example suture implanter during loading with the example suture segment of FIG. 59.

FIG. 61 is a bottom perspective view of the suture implanter of FIG. 60 loaded with the example suture segment of FIG. 59.

FIG. 62 is a bottom perspective view of the suture implanter of FIG. 60 with needles at a first implantation depth and without a loaded suture segment.

FIG. 63 is a top perspective view of an example suturing system.

FIG. 64 is bottom view of the example suturing system of FIG. 63 during loading of an example suture implanter with an example suture segment.

FIG. 65 is a sectional view of the example suturing system of FIG. 64 prior to loading of the one piece suturing segment.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF EXAMPLES

Disclosed are example suturing systems for applying a suture across the tissue break or wound opening. The example suturing systems implant individual spaced suture segments across the tissue break or wound opening. The example suturing systems penetrate tissue on opposite sides of the tissue break or wound opening with a pair of prongs carrying ends of a suture segment to implant the opposite ends of the suture segment. The example suturing systems press those central portions of the suture segments between the implanted ends against a top of the tissue across the tissue break or wound opening. The example suturing systems continue to press the central portions of the suture segments against a top of the tissue across the tissue break or wound opening as the prongs are withdrawn from the tissue to inhibit the implanted ends from being lifted with the prongs being withdrawn and to maintain the implanted ends of the suture segment in place in the tissue.

Disclosed are suturing systems that achieve concurrent implantation of multiple suture segments across a tissue break or wound opening. Such implantation results in opposite end portions of multiple suture segments being concurrently implanted in tissue on opposite sides of the tissue break or wound opening with central portions of such suture segments extending at or near the surface of the tissue, spanning across the tissue break. The disclosed suturing systems are portable, configured for being handheld and manually manipulated. The disclosed suturing systems are lightweight, have a low level of complexity, and are low cost. Each of such systems may be operated using manually applied force, avoiding the need for batteries or other electrical power sources. In some implementations, each of such systems may include a powered actuator for implanting the suture segments. Each of such systems may be utilized as part of a robotic surgical system. For example, each of such systems may be provided on or incorporated into the end of a robotic arm as an end effector. The disclosed suturing systems provide fast, efficient, reliable and steadfast implantation of a suture across a tissue break or wound opening.

Disclosed are example suturing systems that implant a suture segment using prongs configured to catch barbs of the suture segment. The prongs catch or engage the barbs to apply force to the barbs and to the suture segment to drive a suture segment below underlying tissue. As a result, such suturing systems achieve more secure implantation of the suture segment.

Disclosed are example one piece suture segments that have three or more end portions for implantation into tissue along or about a wound. The end portion may or may not include barbs. In some implementations, the one piece suture segments each comprise a central length, wherein multiple legs or end portions extend from either side of the central length. In some implementations, the one piece suture segments each comprise a hub legs or end portions radially extend. In some implementations, such one piece suture segments maybe implanted using the implanter having multiple corresponding implantation shafts or prongs (provided by a staple or multiple needles) configured to catch barbs of the respective end portions.

For purposes of this disclosure, the term “coupled” shall mean the joining of two members directly or indirectly to one another. Such joining may be stationary in nature or movable in nature. Such joining may be achieved with the two members or the two members and any additional intermediate members being integrally formed as a single unitary body with one another or with the two members or the two members and any additional intermediate member being attached to one another. Such a joining may be permanent in nature or alternatively may be removable or releasable in nature.

For purposes of this disclosure, the phrase “configured to” denotes an actual state of configuration that fundamentally ties the stated function/use to the physical characteristics of the feature proceeding the phrase “configured to”.

For purposes of this disclosure, the term “releasably” or “removably” with respect to an attachment or coupling of two structures means that the two structures may be repeatedly connected and disconnected to and from one another without material damage to either of the two structures or their functioning.

The following disclosure describes suture segments to be implanted. Each of the disclosed suture segments may be biodegradable. For purposes of this disclosure, “biodegradable” means that the segment is dissolvable within the natural fluids found within the human body. For example, in some implementations, the suture segments are dissolvable within a time period of 90 to 120 days, in some implementations 210 days and in other implementations, other periods of time per application requirements, in the natural fluids that exist within the human body. In such implementations, connectors 448 may likewise be formed from the same or a different composition that is biodegradable and/or dissolvable within a time period of 90-120 days, or 210 days, depending on use case. Some materials may dissolve in weeks instead of months. within the natural fluids found within the human body.

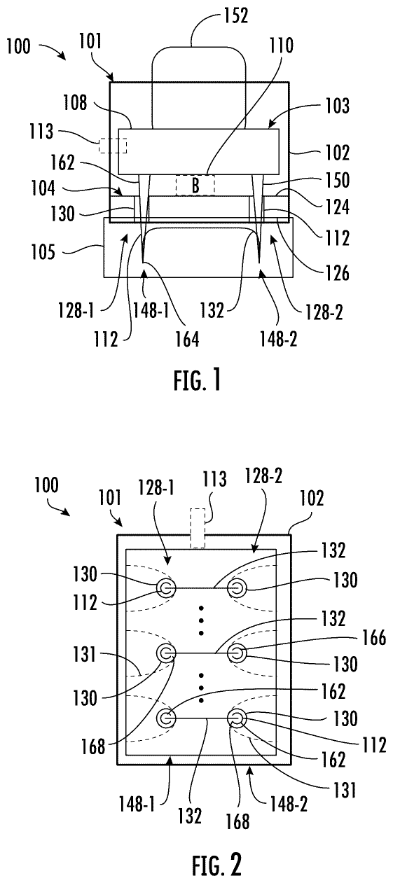

FIGS. 1 and 2 illustrate portions of an example suturing system 100 in a ready loaded state. Suturing system 100 facilitates concurrent implantation of multiple suture segments across a tissue break or wound opening. Such implantation results in opposite end portions of multiple suture segments being concurrently implanted in tissue on opposite sides of the tissue break or wound opening with central portions of such suture segments extending at or near the surface of the tissue, spanning across the tissue break. Suturing system 100 is portable, configured for being handheld and manually manipulated. Suturing system 100 is lightweight, has a low level of complexity, and is low cost. Suturing system 100 may be operated using manually applied force, avoiding the need for batteries or other electrical power sources. Suturing system 100 provides fast, efficient, reliable and steadfast implantation of a suture across a tissue break or wound opening. Suturing system 100 comprises suture applicator or suture implanter 101, guard 105, and suture segments 132.

Suture implanter 101 concurrently implants opposite ends of multiple suture segments 132 into tissue across a tissue break. Implanter 101 comprises housing 102, pressure foot 104 and prong carriage 103. Housing 102 comprises a structure that provides or supports the remaining components of implanter 101. Housing 102 movably or slidably supports prong carriage 103, which is at least partially received within housing 102. Although illustrated as rectangular, housing 102 may have various sizes, shapes and configurations and may movably or slidably support and guide prong carriage 103 in various fashions such as with bearings, tongue and groove configurations and the like.

Pressure foot 104 comprises a structure having a first upper face or side 124 and a second lower face or side 126. Side 126 is configured to press against tissue across the tissue break during implantation of suture segments 132 and as prongs of prong carriage 103 are being withdrawn from the tissue. In some implementations, the surface of the second side 126 is substantially flat. In some implementations, the surface of the second side 126 may be textured or otherwise configured for frictionally gripping and engaging tissue.

As further shown by FIGS. 1 and 2, pressure foot 104 comprises rows 128-1 and 128-2 (collectively referred to as rows 128) of prong passages 130. Rows 128 extend substantially parallel to one another. Each of rows 128 includes at least two prong passages 130, in some implementations, at least three prong passages, and in some implementations, as schematically indicated by ellipses, more than three spaced prong passages 130, wherein each prong passage 130 of row 128-1 is situated across from an associated prong passage 130 of row 128-2. Each prong passage 130 extends from side 124 to side 126. Each prong passage 130 Is sized for the passage of prongs of prong carriage 103 from a first position in which the tips or ends of the prongs extend from the first side 124 past the second side 126 as shown in FIG. 1. In some implementations, each prong passage 130 comprises a circular or oval opening completely surrounded by the body of pressure foot 104. As indicated by broken lines 131, in some implementations, each of prong passages 130 may alternatively comprise a notch extending from a perimeter of the body of pressure foot 104.

In some implementations, pressure foot 104 comprises a plate or other structure mounted to housing 102. In yet other implementations, pressure foot 104 may be integrally formed as part of a single unitary body with housing 102, forming a bottom panel or floor of housing 102. As described hereafter, in yet other implementations, pressure foot 104 may be movable relative to housing 102, within housing 102, and movable relative to prong carriage 103.

Prong carriage 103 comprises body 108 from which rows 148-1 and 148-2 (collectively referred to as rows 148) of prongs 112 project downwardly towards pressure foot 104. Prong carriage 103 is movably or slidably supported within and by housing 102 for movement relative to pressure foot 104. Body 108 comprises a press bar 152 extending through a top of housing 102 or through other portions of housing 102 (such as through a side of housing 102) to facilitate manual raising and lowering of body 108 and prongs 112.

Prongs 112 project from body 108. Each of prongs comprises a shaft 162 having a relatively sharp terminus 164 configured to pierce tissue. The terminus 164 communicates with a hollow interior 166 having a side opening 168 (shown in FIG. 2). The side openings 168 of prongs 112 of row 128-1 face in a first direction while the side openings 168 of prongs 112 of row 128-2 face in a second direction, opposite the first direction, towards the side opening 168 of the associated prong 112 of row 128-1. Each of the side openings 168 may be in the form of a vertical slot having a width sufficiently sized to receive an end portion of a suture segment 132.

As shown by FIGS. 1 and 2, prongs 112 and side 126 of pressure foot 104 cooperate to form, define or retain the general inverted U-shape of suture segment 132 prior to and during implantation of the ends of the suture segment 132 into the tissue across the tissue break. Side 126 bears against the central portions of each of suture segments 132 while each pair of prongs 112 in rows 128-1 and 128-2 bear against the outside edges of the end portions of suture segments 132 that face away from one another such at the end portions extend oblique or perpendicular to the general axis along which the central portions extend. This cooperation facilitates the use of suture segments 132 which are flexible or bendable. This cooperation facilitates the use of suture segments 132 that may be biodegradable or that naturally decompose without the need for subsequent intentional removal following healing.

Although prongs 112 cooperate with side 126 of pressure foot 104 to define the shape of the suture segment during implantation and to retain such shape during implantation, because carriage 103 is movable relative to pressure foot 104, the pressure foot 104 may continue to press the central portions 135 of the suture segments against a top of the tissue across the tissue break or wound opening as the prongs 112 are withdrawn from the tissue to inhibit the implanted ends from being lifted with the prongs 112 being withdrawn and to maintain the implanted ends of the suture segment 132 in place in the tissue. In some implementations, prong carriage 103 is movable relative to pressure foot 104 through manual manipulation of press bar 152.

In some implementations, prongs 112 are integrally formed as part of a single unitary body with body 108 to form prong carriage 103. In some implementations, prongs 112 are releasably and removably connected to body 108, facilitating exchange of prongs 112. In such implementations, prongs 112 may be exchanged for longer or shorter prongs to adjust implantation depth. In such implementations, some of prongs 112 may be omitted to adjust a throw length of the suture (the spacing between opposing pairs of prongs or to adjust the length of the suture (the distance between the end most pairs of prongs).

In some implementations, prong carriage 103 may be formed by a body having bores into which needles (having shafts proximate the terminus which are hollow and have side openings or slots) are inserted and from which the shafts of such needles extend. The needles may be fixedly or permanently mounted within the bores or may be removable from the bores. In some implementations, the prong carriage may comprise a stack of sections with each section comprising a pair of prongs for the two rows of prongs. In some implementations, each of the sections may comprise a plate, wherein the prongs are provided near opposite edges of the plate. In some implementations, the prongs may be formed by integral extending portions of the plate. In some implementations, the prongs may be formed by needles which are mounted to side edges, within side channels or within side of bores of the plate.

As shown by broken lines, in some implementations, implanter 101 may additionally comprise a bias 110 (schematically illustrated) to resiliently bias prong carriage 103 towards a position in which prongs 112 are withdrawn or retracted from projecting beyond side 126. For example, in some implementations, bias 110 may comprise one or more compression springs captured between body and pressure plate 104 or captured between body 108 and a lower upwardly facing surface of housing 102. In such implementations, a person may manually lower prong carriage 103 towards pressure foot 104 against the bias direction of bias 110, wherein reducing the downward force applied to press bar 152 or releasing press bar 152 results in the bias 110 lifting or moving prong carriage 103 away from pressure foot 104. In other implementations, bias 110 may comprise a tension spring connected between an upper side of body 108 and a ceiling of housing 102.

As indicated by broken lines, in some implementations, prong carriage 103 may be releasably retained against pressure foot 104 by a retainer 113. Retainer 113 secures body 108 relative to housing 102 at a selected position. In one implementation, retainer 113 may comprise a spring-loaded pushpin carried by body 108 and popping through an opening within housing 102 to retain prong carriage 103 at a selected position against the bias direction of bias 110. pressing the pushpin against it spring beyond the opening of housing 102 may enable bias 110 to slide or otherwise move prong carriage 103 away from the bottom of housing 102 and away from pressure foot 104, causing prongs 112 to snap back to a position retracted or withdrawn from side 126. In some implementations, the retainer 113 may be omitted. In some implementations, the bias 110 may be omitted such as where manipulation of press bar 152 is solely used to control the positioning of prong carriage 103.

Guard 105 comprises a structure which shields the terminus 164 of each of prongs 112 when implanter 101 is in the state shown in FIG. 1. Guard 105 is removably connected to housing 102 by snaps, pins, a press fit or the like. In some implementations, guard 105 may comprise an annular ring (having an open bottom) releasably mounted to housing 102 and extending from a bottom of housing 102 beyond the terminus 164 of each of prongs 112. In some implementations, guard 105 may comprise an inverted cap releasably connected to housing 102, extending beyond and having a floor that covers or spans across the terminus 164. In yet other implementations, guard 105 may comprise a block of foam or other material into which prongs 112 pierce and penetrate while containing and supporting suture segments 132. In some implementations, guard 105 may be omitted.

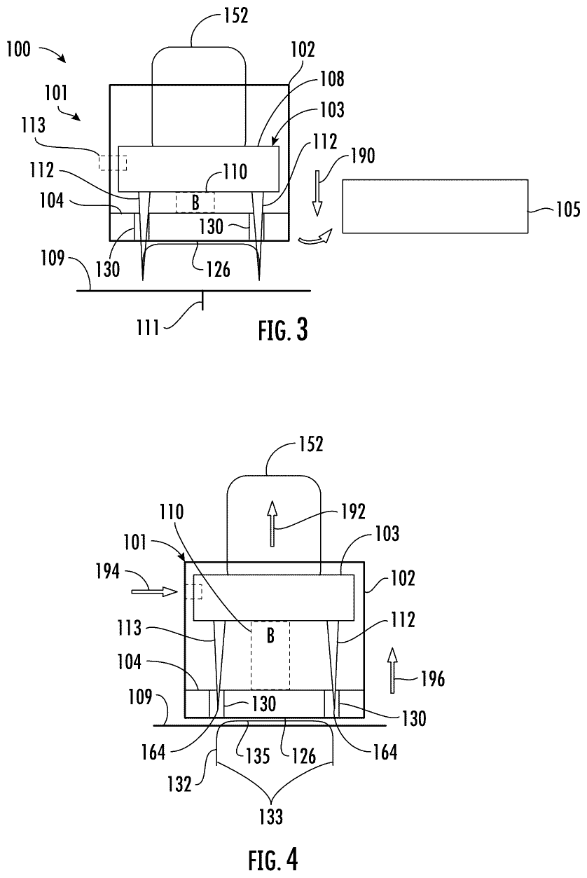

FIGS. 3 and 4 illustrate one example use of implanter 101 during the implantation of suture segments 132 into tissue 109 across a tissue break 111. As shown by FIG. 3, guard 105 (when provided) is separated from housing 102. Thereafter, as indicated by arrow 190, housing 102 and the retained prong carriage 103 are lowered or pressed towards tissue 109 until side 126 of pressure foot 104 is brought into contact with the surface of tissue 109 across tissue break 111. At the same time, the ends of prongs 112 pierce the surface of tissue 109. As a result, the opposite end portions of each of the suture carried by prongs 112 are implanted into and below the surface of tissue 109 on opposite sides of tissue break 111. The central portions 135 of each of suture segments 132 are pressed by side 126 against the surface of tissue 109 across tissue break 111.

As shown by FIG. 4, once the prongs 112 have been fully pressed into tissue 109, implanting the ends 133 of segments, prong carriage 103 is raised in the direction indicated by arrow 192 relative to and away from pressure foot, retracting or withdrawing terminus 164 through passage 130 from side 126 so as to not project beyond side 126. While prongs 112 are being withdrawn, the pressing of side 126 against tissue 109, with the central portions 135 of segments 132 being sandwiched between side 126 and tissue 109, continues to inhibit end portions 133 from being pulled out of tissue 109 despite the lifting of prongs 112.

In some implementations, prong carriage 103 may be lifted through mere manipulation of press bar 152, lifting of press bar 152 relative to housing 102. In implementations where bias 110 is provided, bias 110 may lift or assist in the lifting of prong carriage 103. In implementations where retainer 113 is provided, manual depressant of retainer 113 (in the direction indicated by arrow 194) permits bias 110 to automatically raise or lift prong carriage 103 (and its prongs 112) away from pressure foot 104 towards the top of housing 102. Once the prongs 112 are retracted from side 126, the entire implanter 101 may be lifted away from tissue 109 as indicated by arrow 196.

FIGS. 5-10 are sectional views schematically illustrating an example suturing system 200. Suturing system 200 is similar to suturing system 100 described above except that suturing system 200 comprises implanter 201 in place of implanter 101. Implanter 201 is similar to implanter 101 except that implanter 201 comprises pressure foot 204 in place of pressure foot 104. Those remaining components of system 200 which correspond to components of system 100 are numbered similarly and/or are shown and described above with respect to FIGS. 1-4.

Pressure foot 204 is movable relative to housing 102 and prong carriage 103. In some implementations, housing 102 includes channels or grooves which guide vertical sliding movement of pressure foot 204 within housing 102. As with pressure foot 104, pressure foot 204 comprises rows 128-1 and 128-2 of prong passages 130 extending from side 124 to side 126. As shown by FIG. 7, a handle 206 is connected to and extends from pressure foot 204 through a side opening or slot 219 in housing 102. Handle 206 facilitates manual positioning of pressure foot 204 relative to housing 102 and relative to prong carriage 103.

FIG. 5 illustrates implanter 201 in an unloaded state with prong carriage 103 in a position such that prongs 112 are retracted from and do not extend beyond side 126 of pressure foot 204. In the illustrated state, prong carriage 103 is fully raised (moved away from the bottom of housing 102) while pressure foot 204 is lowered with surface 126 flush with or projecting beyond the bottom of housing 102. As discussed above, in implementations where implanter 201 comprises bias 110, bias 110 may assist in resiliently urging prong carriage 103 to the illustrated retracted position. In implementations where bias 110 is captured between side 124 of pressure foot 204 and a surface of body 108, bias 110 may additionally urge pressure foot 204 to the lowered tissue pressing position shown. In some implementations, bias 110 may not apply a force to pressure foot 204, wherein pressure foot 204 is manually held in position through manipulation of handle 206. In some implementations, bias 110 may be omitted.

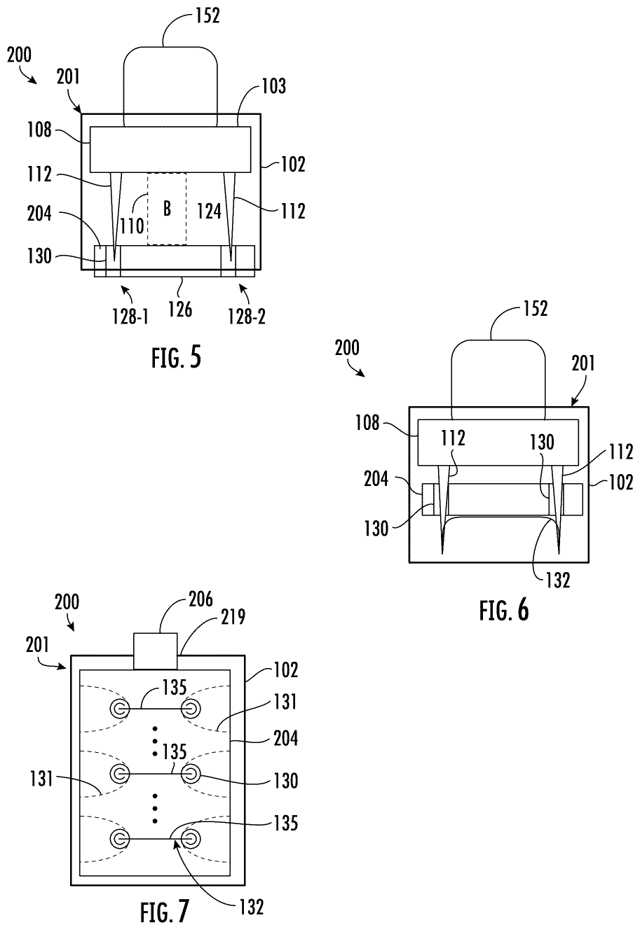

FIGS. 6 and 7 illustrate implanter 201 in a ready and loaded state. To load implanter 201 with suture segments 132, implanter 201 may be inverted (from the orientation shown in FIG. 5) and individual suture segments 132 may be laid across side 126, extending across paired passages 130 of rows 128. In some implementations, the surface of side 126 may include grooves or channels extending between the paired passages 130 to assist in aligning and retaining the suture segments during loading. In yet other implementations, the surface of side 126 may include a pressure sensitive adhesive for temporarily holding suture segments which are stuck and held in place on side 126.

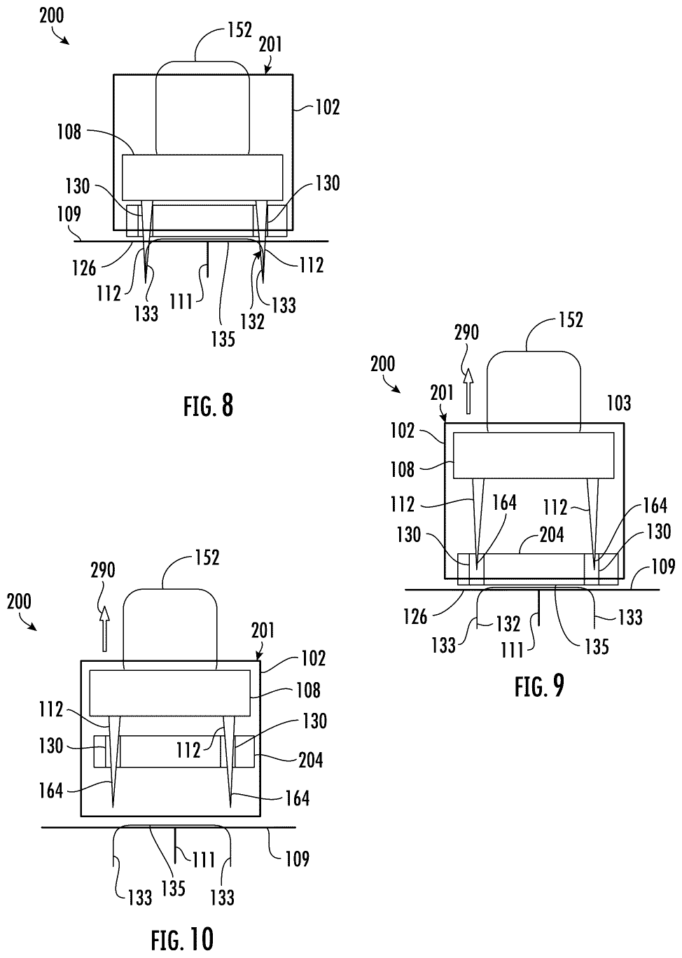

Once the suture segments 132 have been positioned across the face of side 126 and across the paired passages 130, force may be applied to press bar 152 to extend prongs 112 past side 126 while pressure foot 204 is in the tissue pressing position shown in FIG. 5. As a result, the flexible suture segments 132 are bent at their ends so as to have a generally U-shaped, with the ends of the suture segments being received within the hollow interiors of the shafts of prongs 112. This results in system 200 having a configuration similar to that shown in FIG. 8 (without the tissue 109 or tissue break 111). Thereafter, pressure foot 204 and prong carriage 103 (along with the secured suture segments 132) may both be raised (moved away from the bottom of housing 102) to achieve the ready and loaded state shown in FIG. 6.

In some implementations, bias 110 extends between housing 102 and prong carriage 103 to lift prong carriage 103 while pressure foot 204 is manually lifted through manipulation of handle 206. In implementations where bias 110 is captured between body 108 and pressure foot 204, pressure foot 204 is also lifted against bias 110 to the raised position. In some implementations, there is sufficient friction between prongs 112 and the interiors of passages 130 such that lifting of prong carriage 103 (using press bar 152 and/or by bias 110) also raises or lifts pressure foot 204 in substantial unison with the raising of prong carriage 103.

FIGS. 8-10 illustrate the securement of a suture to tissue 109 across tissue break 111. As shown in FIG. 8, the bottom of housing 102 is positioned against in contact with the top of tissue 109 such that the two rows 128 extend on opposite sides of the tissue break 111 being sutured. Manual force is applied to press bar 152 to lower prong carriage 103 and prongs 112. During lowering of prong carriage 103, the bottom of body 108 makes contact with the top side 124 of pressure foot 204, driving pressure foot 204 towards the bottom opening of housing 102 in unison with the lowering of body 108. This lowering movement continues until pressure foot 204 is brought into contact with tissue 109 and prongs 112 have been moved through passages 130, extending beyond side 126 and piercing and penetrating tissue 109, implanting the opposite outer ends 133 of suture segments 132 below the surface of tissue 109 on opposite sides of tissue break 111. At such times, surface 126 is pressing central portions of each of the suture segments 132 against the surface of tissue 109.

As shown by FIG. 9, once the ends 133 of suture segments 132 have been implanted in tissue 109, prongs 112 are withdrawn or retracted in the direction indicated by arrow 290 away from the bottom of housing 102. Such retraction continues until the lower tips are terminus 164 of prongs 112 have been raised or moved so as to no longer project beyond side 126. Even though prongs 112 have been withdrawn, surface 126 may continue to press central portions of each of suture segments 132 against the top of tissue 109. In some implementations, bias 110 may assist in lifting prong carriage 103 while handled 206 is manually grasped to hold pressure foot 204 in the tissue pressing position shown. As shown by FIG. 10, once the prongs 112 have been sufficiently withdrawn, raised above retracted above side 126, pressure foot 204 may be manually lifted using handled 206 to the tissue disengagement position shown in FIG. 10, received within housing above the bottom opening of housing 102. At such times, implanter 101 may be lifted and reloaded with suture segments 132 as described above.

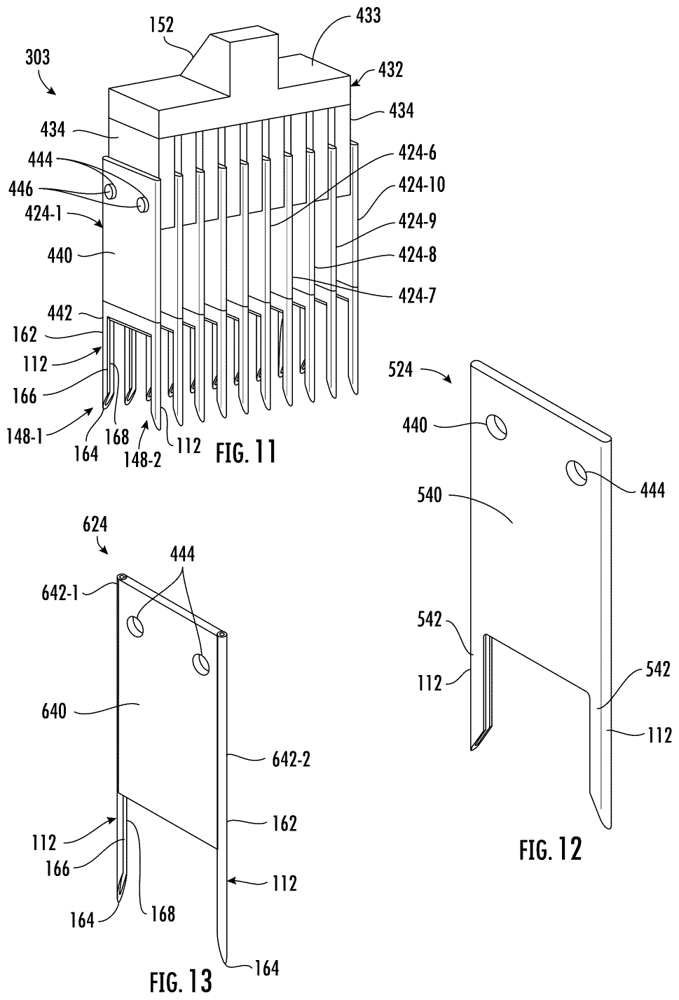

FIG. 11 illustrates an example prong carriage 303 that may be utilized in place of any of the prong carriages described above or hereafter (with modifications to the pressure foot to accommodate the number of pairs of prongs provided by prong carriage 303). Prong carriage 303 comprises a stack of carriage sections 424-1, 424-2, 424-3, 424-4, 424-5, 424-6, 424-7, 424-8, and 424-10 (collectively referred to as sections 424) which are joined and longitudinally spaced from one another by a section holder or carrier 432.

Carrier 432 comprises a body having a top 433 from which press bar 152 upwardly projects and a series of retention arms 434 which are interleaved between sections 424 to longitudinally spaced sections 424. In the example illustrated, arms 434 are releasably connected to sections 424 such that sections 424 may be removed, replaced and/or exchanged to provide different implantation depths, different throw lengths or different suture lengths.

Sections 424 each comprise a central body in the form of a plate 440 supporting an inverted U-shaped staple 442 along its lower edge. Each staple 442 provides the first prong 112 of row 148-1 and the second prong 112 of row 148-2. As described above, each of prongs 112 has a shaft 162 extending to a terminus 164 (a point or edge) which is sufficiently sharp for piercing tissue. Each shaft 162 has a hollow interior 166 having a side opening 168. The side openings 168 of the pair of prongs 112 provided by each section 424 face one another.

In some implementations, each staple 442 is separately formed and welded, bonded or mounted to the lower edge of its respective plate 440. In the example illustrated, each plate 440 comprises a pair of openings 444 which are aligned with corresponding openings in each of arms 434 and with the openings 444 of the remaining plates 440 of sections 424. A pair of pins 446 are passed through the lined openings and secured to releasably mount the selected sections 424 to arms 434 for prong carriage 303. In other implementations, the sections 424 may be releasably mounted to carrier 432 in other fashions. In some implementations, the sections 424 may alternatively be fixed to arms 434 of carrier 432, such as with welding, with bonding or with mechanical interconnections or fasteners.

FIG. 12 illustrates an example probe carriage section 524. Carriage section 524 may be utilized in place of any of sections 424 described above in probe carriage 303. Section 524 comprises a plate 540 having extensions 542 which form prongs 112 (described above). Extensions 542 are integrally formed as part of a single unitary body with plate 540. Plate 540 additionally comprises the above-described pair of openings 444 for releasable mounting of section 524 to carrier 432.

In some implementations, section 524 may be formed by stamping a sheet of material, such as a metal, in the shape shown in FIG. 12 and milling along the inside portion of extension 542 to form a hollow interior 166 and the side openings 168 of prongs 112. In some implementations, section 524 may be formed by a stack of three plates welded, bonded or otherwise joined to one another, wherein the middle plate has shorter width extensions 542 to form an inwardly facing channel along extensions 542 and between the outermost plates to form the hollow interiors 166 and the side openings 168. In still other implementations, section 524 may be molded or cast with the above-described shape.

FIG. 13 illustrates an example probe carriage section 624. Carriage section 624 may be utilized in place of any of sections 424 described above in probe carriage 303. Section 624 comprises plate 640 and a pair of slotted needles 642-1 and 642-2 (collectively referred to as needles 642). Needles 642 are secured on or proximate to opposite side edges of plate 640 so as to extend her project beyond the lower edge of plate 640, forming the pair of prongs 112 for rows 148-1 and 140-2 of prong carriage 303. The lower end of the shaft 162 of each of needles 642 comprises the above-described tissue piercing terminus 164, the hollow interior 166 and the side slot or side opening 168. The needles 642 are oriented such that the side openings 168 of the two needles face one another when mounted or otherwise secured to plate 640. In some implementations, the needles 642 are welded, bonded or otherwise joined to the outer edges of plate 640. In some implementations, plate 640 may have a thicker dimension and may include side grooves along its opposite edges into which needles 642 are inserted and secured. In some implementations, plate 640 may have a thicker dimension and may include a pair of side bores into which needles 642 are inserted and secured.

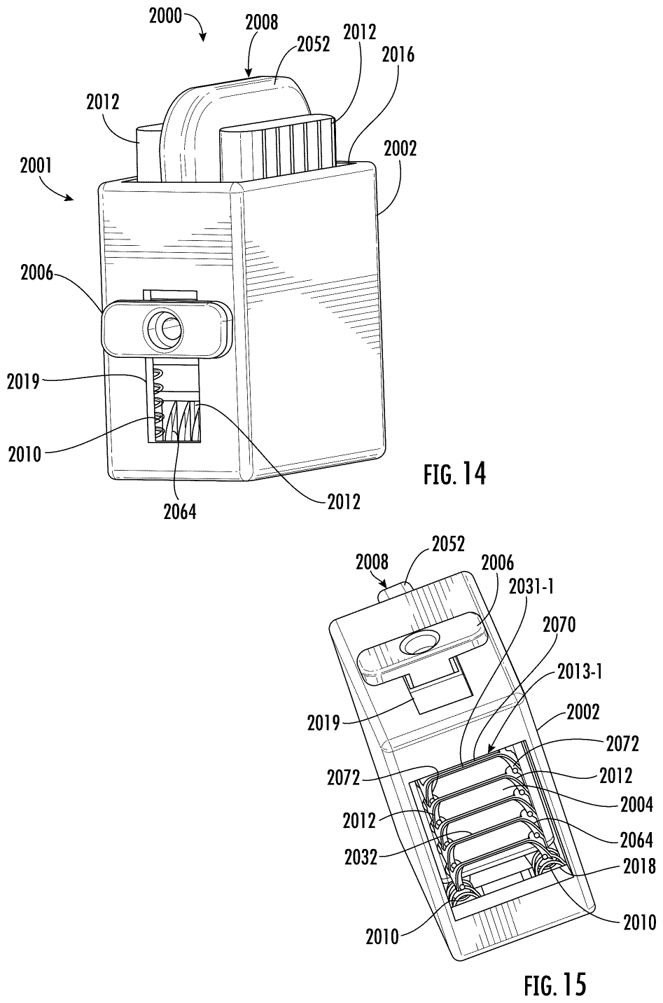

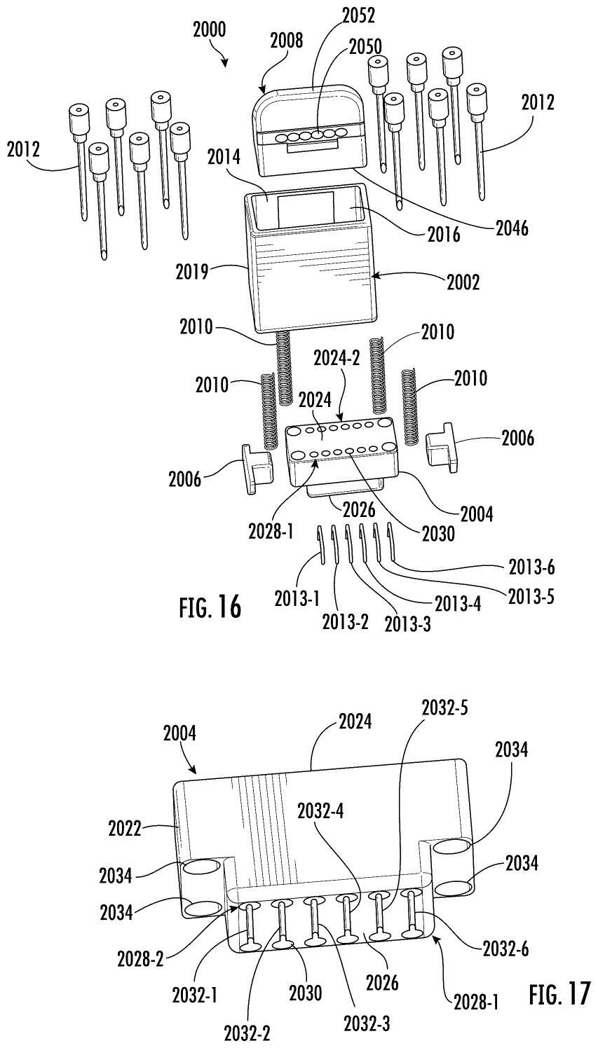

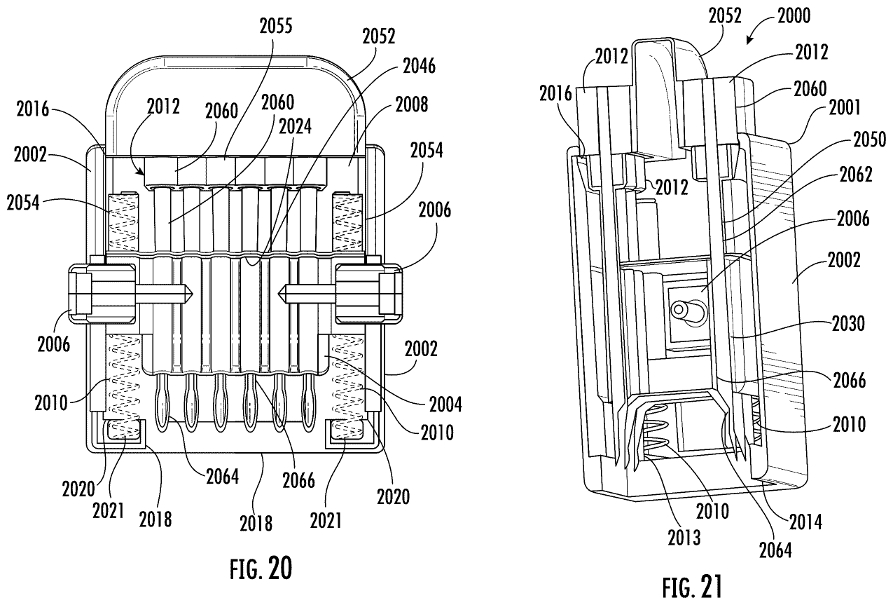

FIGS. 14-21 illustrate portions of an example suturing system 2000. Suturing system 2000 is configured to concurrently implant a plurality of suture segments across a wound opening. FIGS. 14 and 15 illustrate system 2000 in a loaded ready state. FIGS. 16-19 illustrate the various components of system 2000. FIGS. 20 and 21 are sectional views of system 2000 while in the loaded and ready state. As shown by FIGS. 14-16, system 2000 comprises an outer shell or housing 2002, pressure foot 2004, handles 2006, needle holder 2008, bias members 2010, slotted needles 2012 and suture segments 2013-1, 2013-2, 2013-3, 2013-4, 2013-5 and 2013-6 (collectively referred to as suture segments 2013). Needle holder 208 and the slotted needles 2012 form a prong carriage 2003.

Housing 2002 has an interior 2014 that slidably receives pressure foot 2004 and needle holder 2008. Although illustrated as rectangular, housing 2002 may have other sizes and shapes. As shown by FIG. 16, housing 2002 has a top opening 2016. As shown by FIG. 15, housing 2002 has a bottom opening 2018. As shown by FIG. 14, housing 2002 comprises a side opening 2019 (and another side opening on the opposite side of housing 2002) through which handles 2006 project, and along which handles 2006 are slidable.

As shown by FIG. 14, housing 2002 has an interior shoulder 2020 on opposite sides of bottom opening 2018 and bias member bores 2021. Shoulder 2020 comprises blind openings in which lower ends of bias members 2010 are captured and received. In other implementations, bores 2021 may be omitted where other mechanisms are utilized to retain lower ends of bias members 2010 in place.

Pressure foot 2004 is slidably received within interior 2014 proximate to bottom opening 2018. As shown by FIGS. 16 and 17, pressure foot 2004 comprises a body 2022 having an upper surface 2024 and a lower press face 2026. Foot 2004 further comprises rows 2028-1, 2028-2 of needle passages 2030,

Channels 2032 extend between and connect opposite paired needle passages 2030 of rows 2028-1 and 2028-2. Channels 2032 comprise grooves sized to receive and retain a center portion of a suture segment 2013. As will be described hereafter, channels 2032 cooperate with slotted ends of slotted needles 2012 to form a suture segment receiving staple, wherein channels 2032 form a central or center portion of the staple and suture segments center channels 2032-1, 2032-2, 2032-3, 2032-4, 2032-5 and 2032-6 (collectively referred to as channels 2032), and bias bores 2034. Needle passages 2030 extends from upper surface 2024 to lower press face 2026. Needle passages 2030 of rows 2028 are arranged in pairs located on opposite ends of respective channels 2032.wherein the pair of needles 2012 extending through the pair of passages 2030 to form end portions, legs or prongs of the functional suture receiving staple.

In some implementations, channels 2032 may be omitted. For example, in some implementations, lower press face 2026 may alternatively comprise a resiliently compressible surface that partially deforms about a center portion of a suture segment or grips a suture segment. In some implementations, lower press face 2026 may be roughened to freshly contact and retain center portions of suture segments 2013. In some implementations, lower press face 2076 may be incompressible and/or smooth without any special features or material compositions to assist in retaining or gripping the center portion of a suture segment.

Bias bores 2034 extend through body 2022 and are located and sized for receiving bias members 2010. Bores 2034 facilitate bias members 2010 being captured between an interior face of housing 2002 and opposite lower face of needle holder 2008 such that bias members 2010 resiliently bias needle holder 2008 towards top opening 2017 and away from pressure foot 2004. In the example illustrated, bias members 2010 comprise compression springs. In other implementations, bias members 2010 may comprise other forms of springs and may be located at other locations.

Handles 2006 extend from opposite sides of body 2022 through side openings 2019 of housing 2002. Handles 2006 are slidable within side openings 2019 and facilitate manual sliding and movement of pressure foot 2004 within the interior 2014 of housing 2002. Handles 2006 further facilitate manual retention of pressure foot 2004 and a lowered state, flush with or projecting beyond the bottom opening 2018 of housing 2002 to retain suture segments 2013 in place within underlying tissue as needles 2012 are withdrawn (as will be described hereafter).

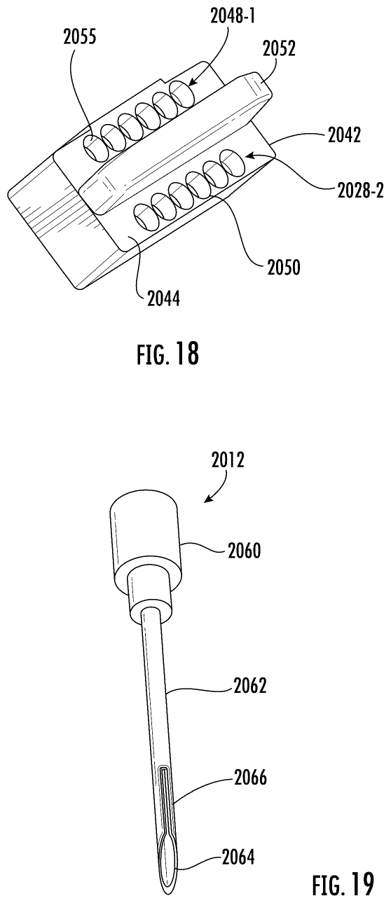

Needle holder 2008 removably or releasably retains and holds one or more individual needles 2012. Needle holder 2008 is slidable up and down within interior 2014 of housing 2002 to move needles 2012 between extended and retracted positions relative to bottom opening 2018. As shown by FIGS. 16 and 18, needle holder 2008 comprises body 2042, top face 2044, bottom face 2046, rows 2048-1, 2048-2 of needle bores 2050, bias member bores 2054 (shown in FIG. 20) and needle actuator 2052.

Rows 2048 and their individual needle bores 2050 correspond to and coincide with rows 2048 and their individual needle passages 2030, respectively. Needle bores 2050 extend from top face 2044 through bottom face 2026. In the example illustrated, each of needle bores 2050 comprises a countersunk opening 2055 for receiving a wider head portion of each of needles 2012.

Bias bores 2054 comprise blind openings projecting into the lower face 2046 of needle holder 2008 at locations corresponding to bores 2034 of pressure foot 2004. Bias bores 2054 receive and capture upper ends of bias members 2010 to retain bias members 2010. In some implementations, bias bores 2054 may be omitted, or in other attachment mechanisms are used to secure the upper ends of bias members 2010 to needle holder 2008.

Needle actuator 2052 comprises a structure projecting upwardly from body 2042 that is configured to be manually pressed downward, towards the bottom opening 2018, against the bias of bias members 2010. Although illustrated as an elongate bar, needle actuator 2052 may have other sizes, shapes and configurations. In some implementations, needle actuator 2052 may project through side openings or slots in housing 2002.

Needles 2012 are removably received within corresponding needle bores 2050. As shown by FIG. 19, each of needles 2012 comprises a head 2060 and a needle shaft 2062. Head 2060 is wider than shaft 2062 and is removably received within the countersink 2055 while shaft 2062 continues through bore 2050 and is movable through an associated needle passage 2030. Shaft 2062 comprises an angled open tip 2064 from which slot 2066 extends upward towards head 2060. The opening of tip 2064 and slot 2066 are sized to receive a side portion of a suture segment 2013. Needles 2012 are to be positioned within needle bores 2050 with slot 2066 facing channel 2032. In some implementations, the upper ends of bores 2050 and the cross-sectional shape of head 2060 may have matching asymmetric inner and outer shapes, respectively, such that head 2060 may only be inserted into bore 2050 in an orientation such that slot 2066 of shaft 2062 faces and is aligned with a centerline of the adjacent channel 2032 when received within bore 2050.

In the example illustrated, each of needles 2012 is removably received within one of bores 2050 such that needles 2012 may be selectively removed and replaced to adjust the number of needles and the number of suture segments being implanted. Different needles having different lengths may be utilized to provide different suture segment implantation depths. Different needles having different size shafts and different configurations may be utilized depending upon particular circumstances. In yet other implementations, needles 2012 may be fixed or non-removably mounted or carried by needle holder 2008. Although needle holder 2008 is illustrated as supporting two rows of six needles, in other implementations, needle holder 2008 may support or carry additional rows and/or alternative numbers of needles in each row.

FIGS. 14, 15, 20 and 21 illustrates the components of system 2000 assembled to form a suturing implanter 2001 loaded with suture segments 2013. Bias members 2010 are received within bores 2021 and 2054, was only biasing needle holder 2008 towards top opening 2016, biasing needles 2012 to retracted positions within the interior of housing 2002. As shown by such figures, needles 2012 are received within bores 2050 with heads 2060 received within countersink's 2054. Shafts 2062 continue to extend from countersink 2055 through bores 2050 and through passages 2030. Passages 2030 are sufficiently tight with respect to shaft 2062 such that pressure foot 2004 is frictionally engaged by the sides of shafts 2062 of needles 2012 and such that the lifting of needle holder 2008 also results in pressure foot 2004 also being lifted or raised towards top opening 2016, retracting press face 2026 above lower opening 2018 and into the interior 2014 of housing 2002.

Suture segments 2013 may be similar to each of the above described suture segments. In some implementations, each of suture segments 2013 are similar to suture segment 132 described above. Each of suture segments 2013 may comprise a flexible line having a barb proximate a first end and a barb proximate a second end. As schematically illustrated, each of such barbs has pointed tips and at least one widened rear portion to the facilitate penetration into tissue but resist withdrawal of the inserted suture segment 2013 from the tissue.

In some implementations, the flexible line providing suture segment 2013 may comprise multiple spaced barbs. In some implementations, the flexible line providing suture segment 34 may comprise a series of spaced barbs along its entire longitudinal length. For example, the flexible line may have a first plurality of barbs on a first side of and pointing away from a longitudinal center of the flexible line and a second plurality of barbs on a second side of and pointing away from the longitudinal center of the flexible line.

In some implementations, the flexible line forming suture segment 34 has shape memory. In some implementations, the flexible line comprises portions that are elastic, wherein the suture segment 2013 may be stretched during penetration and insertion into the underlying tissue, wherein the suture segment will subsequently retract once in the tissue to draw together opposite edge portions of the cut or incision being sutured. In some implementations, the suture segment 2013 is formed from an organ absorbable and dissolvable material, such as Polydioxanone (PDS), eliminating the need to subsequently remove the suture. In some implementations, the suture segment 2013 has a length of at least 9 mm and no greater than 15 mm. In some implementations, the flexible line forming suture segment 2013 has a diameter or thickness of at least 3-0 (United States Pharmacopeia (USP) numbering system for sutures, (0.25 mm) and no greater than 1 USP (0.4 mm).

As shown by FIG. 15, when loaded, each of suture segments 2013 has a central portion 2070 received within a corresponding one of channels 2032 and opposite end of portions 2072 received through slots 2066 of opposite needles 2012 into the interior of shafts 2062. When loaded into the suturing implanter formed by the remaining components of system 2000, suture segments 2013 have a substantially square inverted U-shaped for spanning a wound opening being sutured.

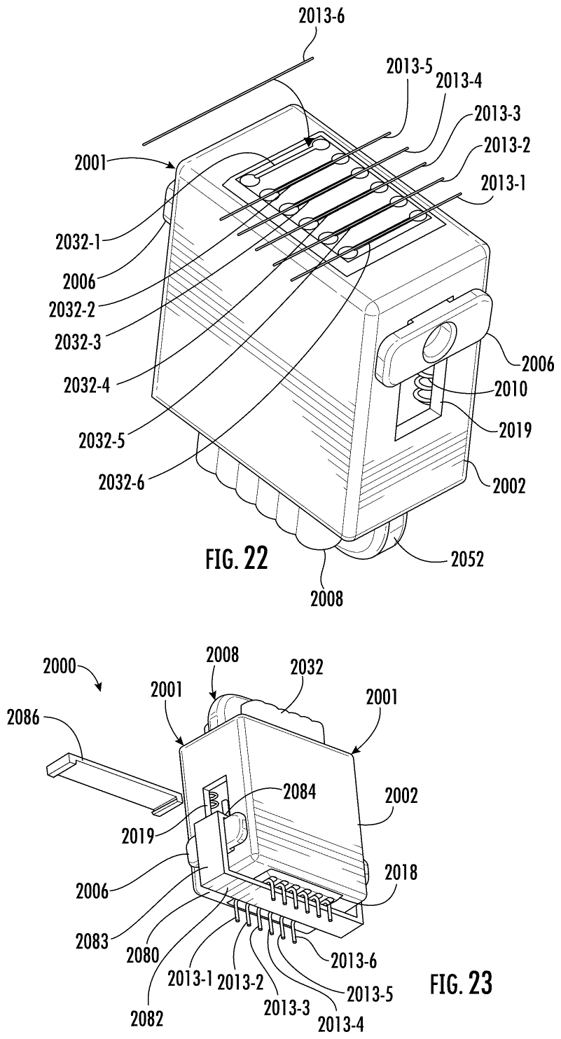

FIGS. 22-27 illustrates one example method by which the suturing implanter of system 2000 may be loaded with suture segments 2013. As shown by FIG. 22, in one possible example loading method, implanter 2001 is inverted and pressure foot 2004 is lifted (moved towards opening 2018) by one manually grasping handles 2006. Pressure foot 2004 is lifted until pressure face 2026 is near, asked, or above bottom opening 2018. This results in pressure foot 2004 also being lifted away from and out of contact with needle holder 2008, which is held in place near top opening 2016 under the force of bias members 2010. It is in part due to pressure foot 2004 freely floating between the bottom side of needle holder 2008 and the internal shoulder surface 2020 of housing 2002. As noted above, in the absence of manual force being applied to pressure foot 2004 via handles 2006, sufficient friction between the shafts 2062 of needles 2012 and the interior surfaces of needle passages 2030 exists such that the raising of needles 2012 and needle holder 2008, such as under the force of bias members 2010, also lifts (movement towards top opening 2016) pressure foot 2004. This friction force between needles 2012 and needle passages 2030 may be overcome with manual force applied to pressure foot 2004 through handles 2006.

As further shown by FIG. 22, suture segments 2013-1 to 2013-6 are laid across and positioned within channels 2032-1 to 2032-6, respectively. Although suture segment 2013 are illustrated as having ends which linearly project outward, depending upon their flexibility, in some implementations, opposite end portions of each of suture segments 2013 may alternatively droop towards and/or along the sides of housing 2002.

Once the suture segments 2013 have been laid in place, they may be secured in place by the positioning of the example retainer 2080 shown in FIGS. 23, 24 and 25. Such positioning may be done while the implanter 2001 is in the inverted state shown in FIG. 22. Thereafter, the implanter 2001 and the secured suture segment 2013 may be restored to the upright positions shown in FIGS. 23-25. In the example illustrated, retainer 2080 comprises a U-shaped member having a bottom 2082 that spans across each of channels 2032 and upwardly extending arms 2083 that extend along the sides of implanter 2001. In the example illustrated, each of arms 2083 comprises an interleague projecting catch or hook 2084 that extends over the top edge of each of handles 2006 to hold handles 2006 and pressure foot 2004 towards bottom opening 2018. In the example illustrated, at least portions of retainer 2080 are sufficiently resiliently flexible (such as having dimensions and being formed from a resiliently flexible polymer or a resiliently flexible springlike metal) such that the arms 2083 may be widened and snapped about handled 2006. In yet other implementations, arms 2083 may be secured to bottom 2082 by mechanical hinges. In other implementations, each of arms 2083 may be secured to and against structural components of implanter 2001 other than handles 2006.

In some implementations, in lieu of retainer 2080, system 2000 may utilize other means for securing suture segments 2013 to pressure face 2026. For example, some implementations, the interior channels 2032 may include a pressure sensitive adhesive making the interior channels sufficiently sticky to temporarily hold suture segments 2013. In some implementations where pressure face 2026 omits channels 2032, the face itself may include a coating or layer pressure sensitive adhesive making such surfaces sufficiently sticky to temporarily holds suture segments 2013. In still other implementations, the lower lips of the channels 2032, along the lower mouths of channels 2032, may include an elastic rubber-like overhang providing a slot having a width less than the width or diameter of suture segments 2013 to retain suture segments 2013 within channels 2032, but wherein application of manual force is sufficient to pull the suture segments 2013 across the one or more lips which flexes the lips permit the suture segments to be inserted into or pulled out of the respective channels 2032.

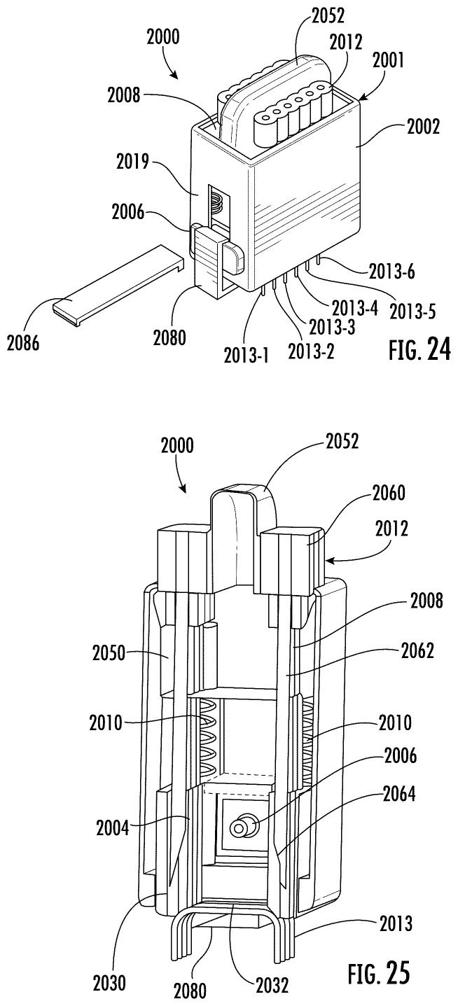

As further shown by FIGS. 23 and 24, in some implementations, system 2000 additionally comprises one or more shims 2086. Shims 2086 are sized to be inserted through side openings 2019 and extend at least partially across the top or upper face 2024 of pressure foot 2004 as shown in lines in FIG. 25. When positioned in place, shims 2086 are sandwiched between pressure foot 2004 and needle holder 2008 such that the extent that needle holder 2008 may be lowered towards bottom opening 2018 is limited. This results in the extent to which needles 2012 may be lowered beyond bottom opening 2018 also being limited. As a result, shims 2086 control the penetration depth of needles 2012 when a suture is being implanted. In some implementations, system 2000 may comprise multiple different shims 2086 where each of the shims 2086 has a different thickness to provide selectable needle penetration depths.

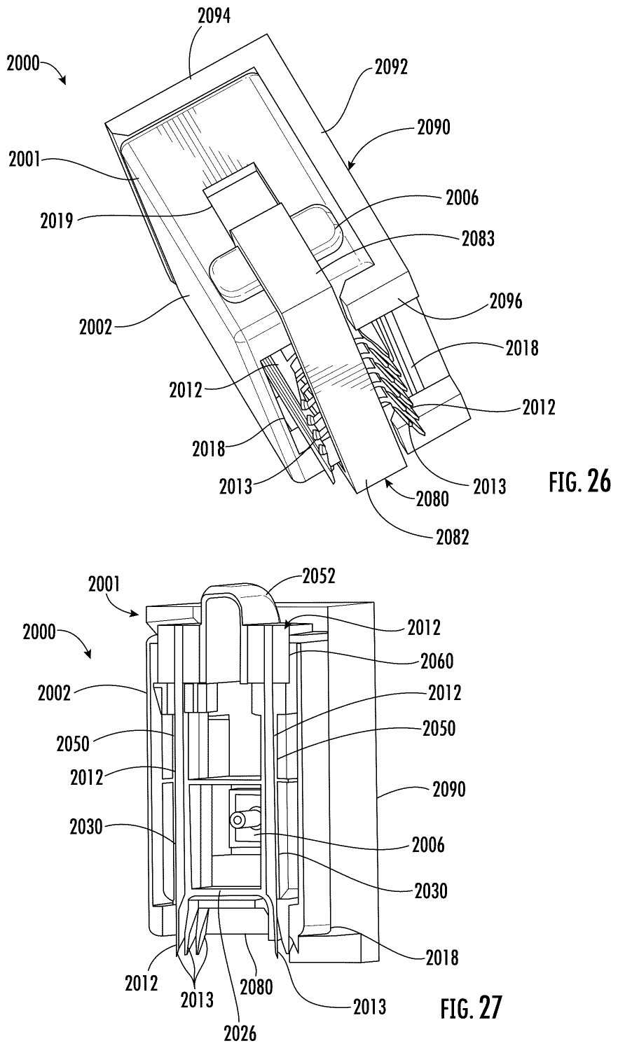

As shown by FIGS. 26 and 27, once the central portions of suture segment 2013 have positioned and secured in place against pressure face 2026 by retainer 2080, the opposite end portions of segment 2013 are positioned within the interiors of the lower ends of respective needles 2012. In the example method, this is achieved by the user manually depressing needle actuator 2052, against a bias of bias members 2010, to move needle holder 2008 towards bottom opening 2018, to lower tip 2064 of each of the needles 2012 beyond pressure face 2026 and beyond bottom opening 2018. While the tip 2064 of needles 2012 are extending beyond bottom opening 2018, the opposite end portions of each of segment 2013 may be inserted through slot 2066 into the interior of needle shaft 2062 of each of needle 2012. In some implementations, the office in portions of suture segment 2013 naturally move through slot 2066 into the interior of needle shaft 2062.

In some implementations, system 2000 may include retainer 2090 to hold needle holder 2008 and the carried needles 2012 in the extended positions in which needles 2012 project beyond opening 2018. In the example illustrated, retainer 2090 extends along the height or sides of housing 2002, extending over top of needle actuator 2052 to hold needle holder 2008 in a pushed in position with respect to housing 2002.

In the example illustrated, retainer 2090 comprises a U-shaped member comprising a vertical post portion 2092, a top extension 2094 and a bottom extension 2096. Post portion 2092 extends along the sides of housing 2002. Top extension 2094 horizontally extends from post portion 2092 over top of needle actuator 2052. Bottom extension 2096 serves as a hook or catch, rejecting along the bottom of housing 2002 on one or both sides of bottom opening 2018. In the example illustrated, at least portions of retainer 2090 are sufficiently resiliently flexible (such as having dimensions and being formed from a resiliently flexible polymer or resiliently flexible springlike metal) such that the extensions 2094 and/or 2096 may be widened and snapped about housing 2002.

Once each of the end portions of suture segments 2013 have been positioned within the interior of the respective needles 2012, retainer 2080 (when utilized) may be withdrawn from housing 2002. At such time, needle holder 2008, being held by retainer 2090, continues to hold needles 2012 and their extended positions and bears against the top face 2024 of pressure foot 2004 to also hold pressure foot 2004 in its lowered position with pressure face 2026 at or beyond opening 2018 of housing 2002. Thereafter, retainer 2090 may be separated or withdrawn from housing 2002.

Disconnection of retainer 2090 permits bias members 2010 to raise or lift needle holder 2008 back towards the default state proximate to top opening 2016. As discussed above, the friction between each of needle shaft 2062 and the closely conforming needle passages 2030 of pressure foot 2004 frictionally connects needles 2012 to pressure foot 2004 such that pressure foot 2004 move in unison with needle holder 2008 upward towards top opening 2016 in response to disconnection of retainer 2090. Pressure face 2026 with the secured central portions of suture segments 2013 and the lower ends of needles 2012 with the secured opposite end portions of suture segments 2013 are lifted insubstantial unison to retracted or inset positions within housing 2002 (above bottom of 2018) to achieve the loaded and ready state shown and described above with respect to FIGS. 14, 15, 20 and 21.

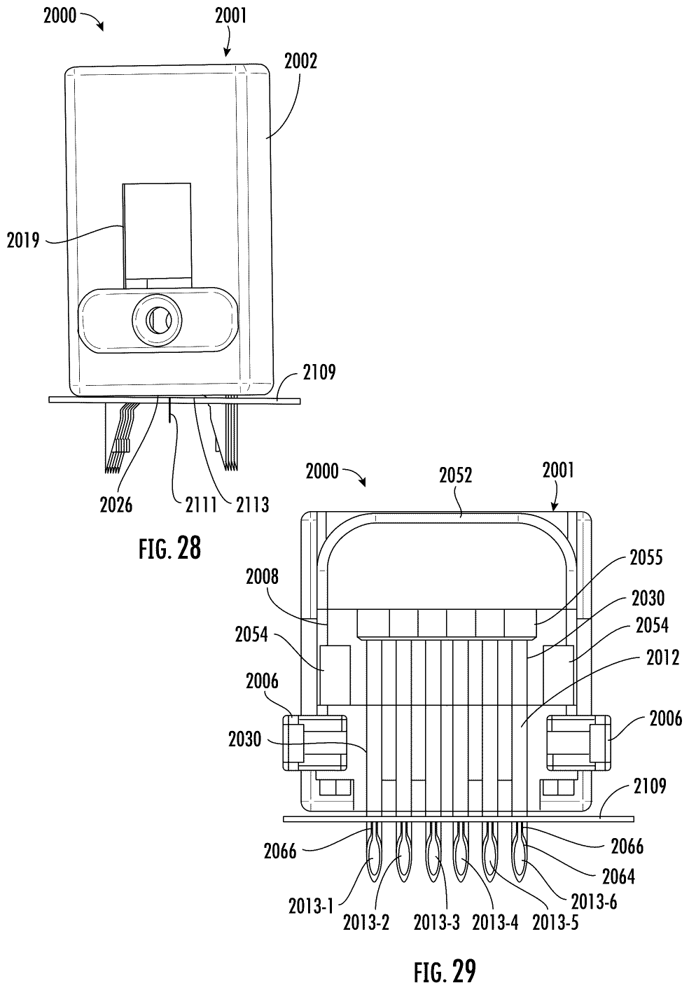

FIGS. 28-34 illustrate the implanting of sutures 2013 by implanter 2001 across a wound opening 2111 in tissue 2109. To suture a wound opening, the loaded and ready implanter 2001 shown in FIGS. 14, 15, 20 and 21 is positioned on tissue 2109 so as to straddle or extend perpendicularly across the wound opening 2111. Once in place, a person may hold or press housing 2002 against the underlying tissue 2109 while applying manual forced to actuator 2052, driving needle holder 2008 towards bottom opening 2018 which results in needle holder 2008 further engaging the top face 2024 of pressure foot 2004 to also concurrently drive pressure foot 2004 towards bottom opening 2018. This downward pressure is applied until pressure face 2026 tissue 2109 on opposite sides of wound opening 2111 and until the lower ends of needles 2012 have penetrated tissue 2109.

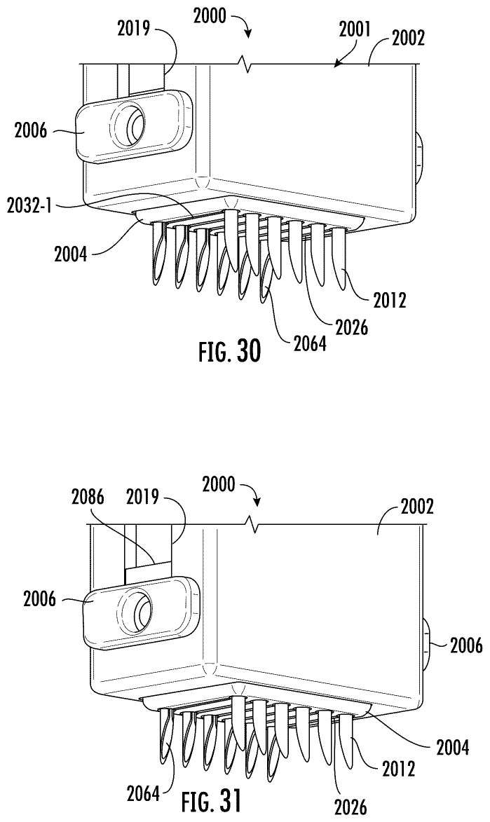

As shown by FIGS. 28 and 29, in this state, central portions of segment 2013 are pressed against or held against the top of surface 2109 by pressure face 2026 of pressure foot 2004. At the same time, the opposite end portions of each of segments 2013 reside within the hollow interior of the respective needles 2012, below the surface of tissue 2109. As shown by FIGS. 28 and 29, the depth at which needles 2012 penetrate the underlying tissue in the depth at which the opposite ends of segments 2013 penetrate underlying tissue may be controlled such as through the use of shims 2086 described above. FIG. 30 illustrates a first insertion depth such as when applicant 2001 use without any shims 2086 while FIG. 31 illustrates a shallower insertion depth or penetration depth such as when shim 2086 is inserted into implanter 2001 as shown by broken lines in FIG. 25.

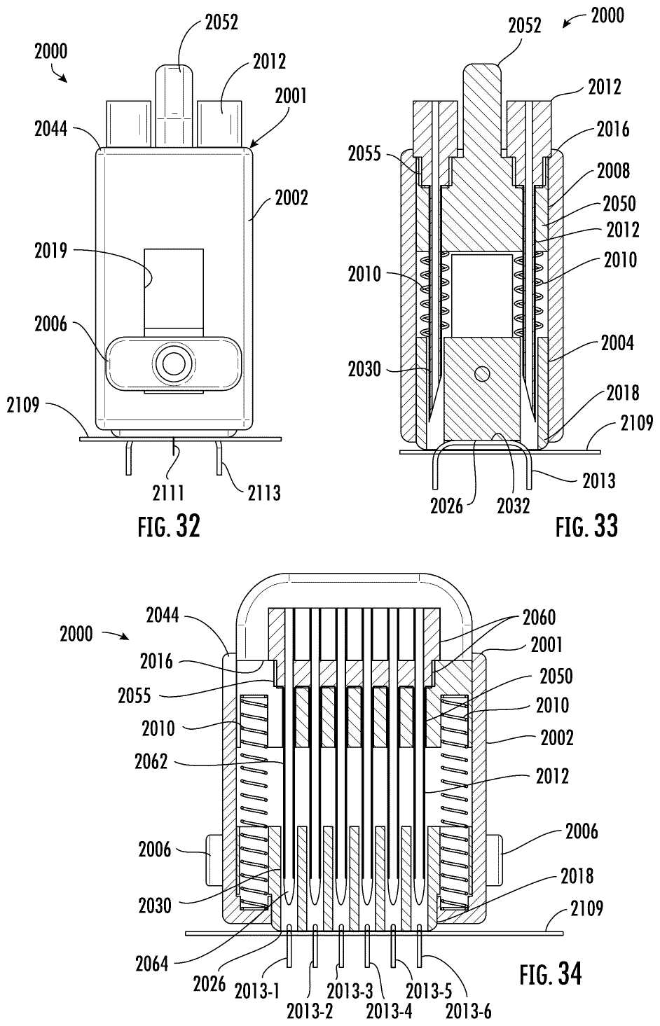

Once the opposite ends of each of suture segments 2013 have been implanted within tissue 2109, needles 2012 and implanter 2001 may be withdrawn from the surface of tissue 2109, leaving suture segments 2013 in place. As shown by FIGS. 32, 33 and 34, following the implantation of needles 2012 with the contained ends of suture segments 2013, the person may reduce the downward pressure applied to needle actuator 2052, permitting bias members 2010 to once again lift or raise needle holder 2008 and needles 2012 back towards top opening 2016. This results in needles 2012 being withdrawn from the underlying tissue 2109 and being moved to the retracted positions above pressure foot 2004 and within housing 2002.

As further shown by FIGS. 32, 33 and 34, to maintain the fully implanted state of suture segments 2013 within tissue 2109 as the needles 2012 are lifted or raised, a user may manually hold down handles 2006 and pressure foot 2004 as needle holder 2008 and needles 2012 are lifted. As a result, as the needles are lifted, pressure face 2026 continues to apply pressure to the tops of each of the suture segments 2013, maintaining their implanted states. Once the lower tips of needles 2012 have been retracted into housing 2002, retracted above pressure face 2026 of pressure foot 2004 (as shown in FIGS. 32, 33 and 34), handles 2006 may be released or manually raised as desired, and implanter 2001 may be lifted from the surface of tissue 2109, readying implanter 2001 for once again being reloaded with suture segments as described above. In some implementations, implanter 2001 may be configured as a disposable, or an implanter that is not reused.

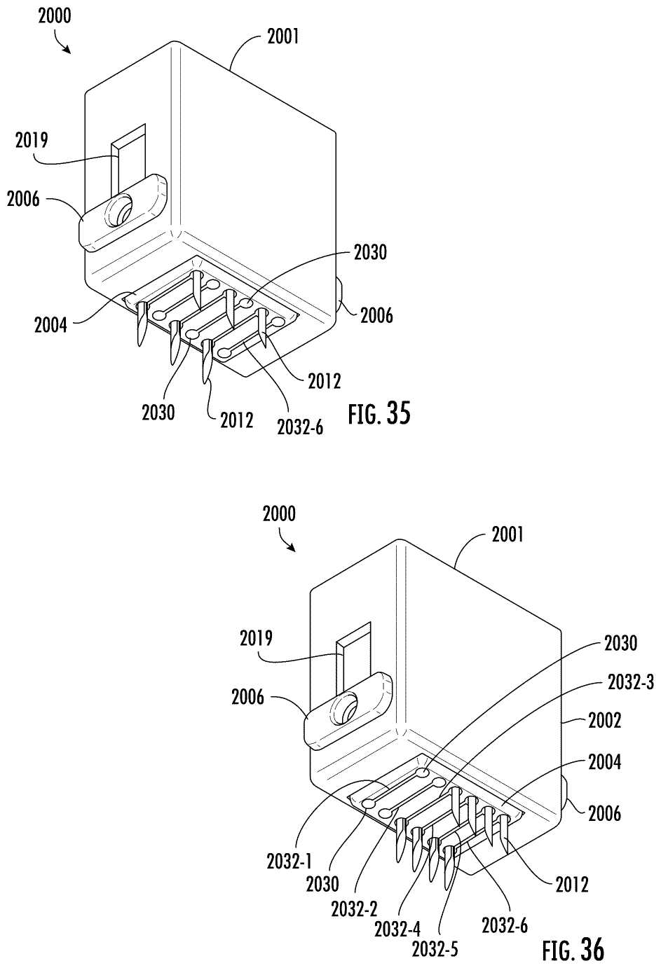

FIGS. 35 and 36 illustrate alternative configurations of implanter 2001. FIG. 35 illustrates an example configuration where particular needles 2012 have been removed from needle holder 2008 to reduce the number of suture segments along the wound opening 2111, increasing the throw spacing between the needle pairs and between the consecutive suture segments being implanted. In the example illustrated, channels 2032-1, 2032-3 and 2032-5, and their associated needle pairs, are occupied while the remaining channels 2032 and their associated needle passages 2030 remain empty. As should be appreciated, other combinations of needle pairs on opposite ends of channels 2032 may be utilized.

FIG. 36 illustrates an example configuration where the closure length of the set of sutures is reduced. In particular, the length between the end most channels 2032 of the series of plant suture segments is reduced. In the example illustrated, the needles 2012 associated with are aligned with the needle past on the opposite ends of channels 2032-1 and 2032-2 are removed, wherein only the last four channels 2032-3, 2032-4, 2032-5 and 2032-6 are loaded with suture segments 2013 and only those particular needles 2012 associated with such channels are mounted or provided within needle holder 2008.

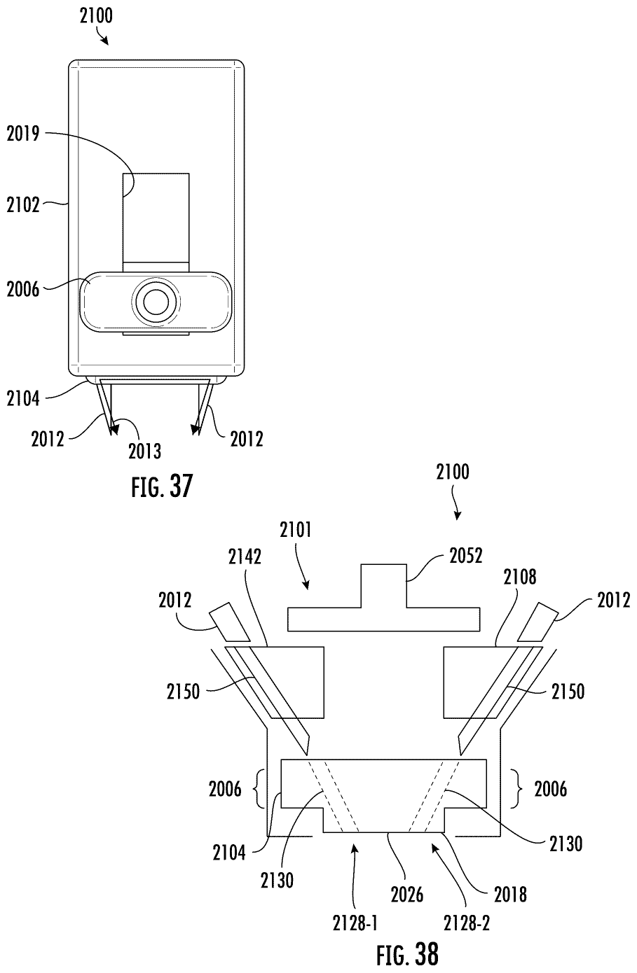

FIGS. 37 and 38 illustrate an example suturing system 2100. Suturing system 2100 is similar to suturing system 2000 described above in all respects except that system 2100 comprises implanter 2101 which comprises housing 2102, pressure foot 2104 and needle holder 2108 in place of housing 2002, pressure foot 2004 and needle holder 2008, respectively. Those remaining components of system 2100 which correspond to components of system 2100 are numbered similarly and/or are shown and described above with respect to system 2000.

Housing 2102 is similar to housing 2002 except that housing 2102 accommodates interior sliding movement of needle holder 2108 which may have a trapezoidal shaped body 2142. Pressure foot 2104 is similar to pressure foot 2004 except that pressure foot 2104 comprises two rows 2128-1 and 2128-2 of needle passages 2130. Such needle passages 2130 are similar to needle passages 2030 except that needle passages 2130 extend along axes that are oblique to the pressure face 2026 of pressure foot 2104, angling towards one another as they approach bottom opening 2018. The number of needle passages 2130 in each of such rows 2182 may be similar to system 2000 or may comprise a greater or fewer of such passages 2130 and a greater or fewer of such rows.

Likewise, needle holder 2108 is similar to needle holder 2008 except that needle holder 2108 comprises needle bores 2150 in place of needle bores 2130. Needle bores 2150 extend within body 2142 and are axially aligned with, coincide with and correspond to needle passages 2130. Needle bores 2150 extend along axes that are oblique to the pressure face 2026 of pressure foot 2104, angling towards one another as they approach bottom opening 2018. In some implementations, needle bores 2150 may include countersunk portions (similar to countersinks 2055) for receiving the heads 2060 of needles 2012. Due to the obliquely extending and downwardly converging rows of needle bores 2150 and needle passages 2130, needles 2012 are implanted at converging angles on opposite sides of the wound or opening being sutured. This may enhance the ability of such sutures to maintain the wound opening and a closed or near close state during healing.

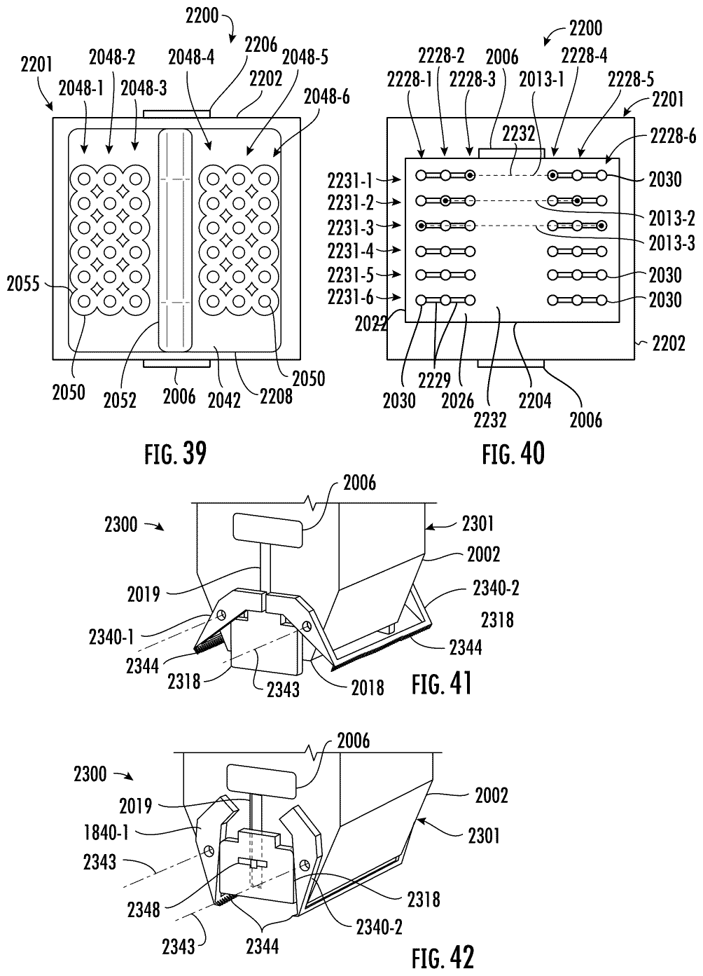

FIGS. 39 and 40 illustrate suturing system 2200. FIG. 39 is a top view of system 2200 while FIG. 40 is a bottom view of system 2200. Suturing system 2200 is similar to suturing system 2000 described above in all respects except that system 2200 comprises implanter 2201 which comprises housing 2202, pressure foot 2204 and needle holder 2208 in place of housing 2002, pressure foot 2004 and needle holder 2008, respectively. Those remaining components of system 2200 which correspond to components of system 2100 are numbered similarly and/or are shown and described above with respect to system 2000. FIGS. 39 and 40 omit needles 2012 and suture segments 2013 (shown and described above).

As with housing 2002, housing 2202 slidably supports and contains pressure foot 2204 and needle holder 2208, with needle holder 2208 supported above pressure foot 2204. As with housing 2002, housing 2202 comprises two side openings through which handles 2006, which extends from pressure foot 2204, extend for manual positioning of pressure foot 2204.

Pressure foot 2204 is similar to pressure foot 2004 except that pressure foot 2204 comprises six rows 2228-1, 2228-2, 2228-3, 2228-4, 2228-5 and 2228-6 (collectively referred to as rows 2228) of needle passages 2030 (described above). In the example illustrated, the needle passages 2030 are also arranged to provide six columns 2231-1, 2231-2 2231-3, 2231-4, 2231-5 and 2231-6 of passages 2030 which define the longest potential length of the suture being formed. Rows 2220-1, 2220-2 and 2228-3 are separated from rows 2228-4, 2228-5 and 2228-6 by channels 2232. Channels 2232 are similar to channels 2032 described above. Channels 2232 comprise grooves cut into face 2026 of body 2022 and are configured to receive central portions of suture segments 2013 (shown and described above).

In the example illustrated, secondary channels 2229 extend between the aligned passages 2030 of rows 2228-1, 2228-2 and 2228-3 as well as between the aligned passages 2030 of channels 2228-4, 2228-5 and 2228-6. Secondary channels 2229 may have the same configuration as that of channels 2232, assisting in positioning (and some implementations, retaining) central portions of suture segments 2013. In yet other implementations, secondary channels 2229 may be omitted.

Needle holder 2208 is similar to needle holder 2008 except that needle holder 2208 comprises six rows 2248-1, 2248-2, 2248-3, 2248-4, 2248-5 and 2248-6 (collectively referred to as rows 2248) of needle bores 2050 (described above). Each of rows 2248 and their respective needle bores 2050 are aligned with and coincide with respective rows 2228 of needle passages 2230. For example, each of the needle bores 2250 of row 2048-1 are axially aligned with the underlying needle passages 2230 of row 2028-1. Each of the needle bores 2050 of the remaining rows 2048 have similar alignments with the needle passages of rows 2028.

Suturing system 2200 provides customized suturing configurations. As described above with respect to FIGS. 35 and 36, different combinations of needles may be inserted into and through needle bores 2050 and needle passages 2030 to provide different throw spacings and different suture lengths. In addition, system 2200 further provides user selectable suture widths. In particular, system 2200 accommodates different lengths of user selectable suture segments 2013 through the selection of different spacings between the mountain needles 2012. For example, as schematically shown by broken lines, in some circumstances, suture segments 2013-1 having a first length may be utilized with needles 2012 within needle passages 2030 of rows 2228-3 and 2228-4. This arrangement may be utilized in each of the columns 2231 of passages 2030, or selected portion of columns 2231 so as to provide the selected throw length or suture length as described above with respect to FIGS. 35 and 64. As schematically shown by broken lines, in some circumstances, suture segments 2013-2 having a second length, longer than the first length, may be utilized with needles 2012 within needle passages 2030 of rows 2228-2 and 2228-2. This arrangement may be utilized in each of the columns 2231 of passages 2030, or selected portion of columns 2231 so as to provide the selected throw length or suture length as described above with respect to FIGS. 35 and 64. In some circumstances, as schematically shown by broken lines, suture segments 2013-3 having a third length, longer than the first length the second length, may be utilized with needles 2012 within needle passages 2030 of rows 2228-1 and 2228-9. This arrangement may be utilized in each of the columns 2231 of passages 2030, or selected portion of columns 2231 so as to provide the selected throw length or suture length as described above with respect to FIGS. 35 and 36. In some circumstances, different lengths of suture segments may be utilized along the length of the suture. For example, the suture segments 2013-1 and 2013-3 may be alternated along columns 2231. Longer suture segments may be utilized in the more central or middle columns 2231 while shorter suture segments are utilized in the end most columns of the suture. In some implementations, different suture segments having properties may be utilized amongst the different columns 2231.

Although system 2200 is illustrated as having six columns and six rows of passages 2030 (and corresponding aligned needle bores 2050), in other implementations, system 2200 may have a greater or fewer number of such rows and/or columns. Although system 2200 is illustrated as being substantially symmetric, with three rows on each side of channels 2232, in other implementations, system 2200 may be asymmetric, with a greater number of rows 2228 on one side of channels 2232 as compared to the other side of channels 2232. Although of the aligned needle passages 2030 and needle bores 2050 and access perpendicular to the pressure face 2026, in other implementations, each aligned needle bore 2050 and needle passage 2130 may extend oblique to pressure face 2026, such as in a manner similar to that shown in FIG. 38.

FIGS. 41 and 42 illustrate suturing system 2300. System 2300 is identical to system 2000 except that system 2300 comprises an implanter 2301 that additionally comprises jaws 2314 and jaw actuators 2318. Those remaining components of system 2300 which correspond to system 2000 are numbered similarly and/or are shown in the above figures with respect to system 2000. As shown by FIG. 41, system 2300 has a slightly reconfigured housing 2002 to accommodate jaws 2314 and jaw actuators 2318.

Jaws 2314 and jaw actuators 2318 cooperate to pinch and/or hold those portions of tissue on opposite sides of the tissue break or wound together as pressure foot 2004 and needle holder 2008 are lowered such that the segments 2013 penetrate the tissue on opposite sides of the wound and release the carried flexible suture lines into the tissue across the wound or tissue break, creating the suture. Jaws 2314 comprise a pair of tissue grippers 2340-1, 2340-2 (collectively referred to as grippers 2340) that are each pivotably connected to housing 2002 for pivoting about axes 2343 between an open state shown in FIG. 41 and a closed shown in FIG. 42. Each of grippers 2340 comprises a row of teeth 2344 to assist in gripping or engaging the underlying tissue.

Jaw actuator 2318 comprises a mechanism configured to actuate grippers 2340 between the open and closed states. In the example illustrated, jaw actuators 2318 comprises plates vertically slidable within slot 2019 to move upwardly and downwardly. As shown by FIG. 42, in one example implementation, jaw actuators 2318 may each comprise a bar or tongue 2348 that slides within a vertical groove provided by slot 2019 in housing 1832. In yet other implementations, jaw actuator 2318 may be vertically slidable within a slot within housing 2002 that is different than slot 2019. As further shown by FIGS. 41 and 42, jaw actuators 2318 are is sized and shaped to physically contact and ride against grippers 2340 above axes 2343 such that upward movement of jaw actuators 2318 relative to axes 2343 pivots grippers 2340 about axes 2343 from the open state shown in FIG. 41 to the closed state shown in FIG. 42.

In some implementations, jaw actuators 2318 (or both of jaw actuator 2318) may be resiliently biased towards the lowered position. For example, in some implementations, jaw actuator 2318 (or jaw actuator 2318) may be resiliently biased by a tension spring having a first and connected to housing 2002 and a second end connected to jaw actuator 2318. In some implementations, grippers 2340 may be resiliently biased towards the illustrated open state shown in FIG. 41, wherein movement of jaw actuators 2318 works against such bias to close grippers 2340. For example, in some implementations, grippers 2340 may be resiliently biased towards the open state by torsion springs having a first end secured to an associated one of grippers 2340 and a second end secured to housing 2002.

In the lowered state, jaw actuators 2318 extend or project below mouth 1822. When housing 1802 is being lowered or move towards the underlying tissue, pressure foot 1818 contacts the underlying tissue first, prior to bottom opening being brought into contact with the underlying tissue or wound. As a result, in response to the bottom of housing 2002 being lowered into contact with and across the underlying wound, the closing of grippers 2340 is at or near completion, gripping opposite sides of a tissue break and potentially pinching opposite sides of the tissue break towards one another for subsequent suturing. In the example illustrated, jaw actuators 2318 are sized and mounted to housing 2002 such that grippers 2340 or in a closed state, squeezing opposite sides of the tissue break together, prior to pressure foot 2004 and needle holder 2008 being fully lowered to implant the suture segments 2013 in the tissue.

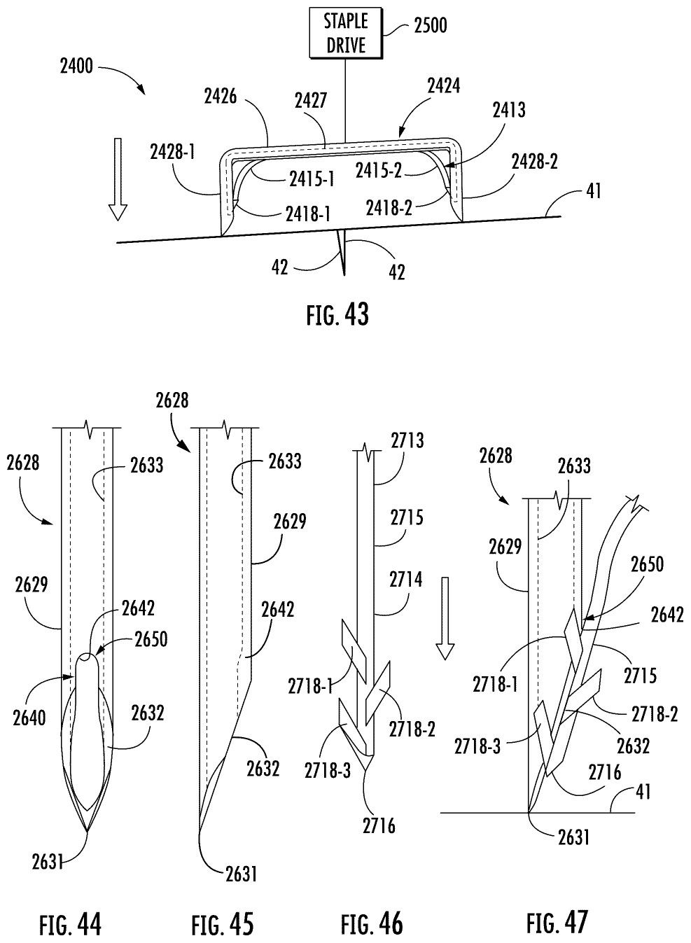

FIG. 43 illustrates portions of an example suturing system 2400 which comprises suture segment 2413, staple 2424 and staple drive 2500. The example suturing segment 2413 comprises a flexible line having a pair of penetrating end portions 2415-1, 2415-2 (collectively referred to as end portions 2415) joined by an intermediate portion 2416. End portion 2415-1 comprises a first barb 2418-1 proximate to a first end to tip of end portion 2415-1. End portion 2415-2 has a second barb 2418-2 proximate to a second end or tip of end portion 2415-2. Each of the barbs 2418-1 and 2418-2 are above the end or tip of the end portion 2415, meaning that the barbs are axially located between both ends or tips of the suture segment 2413. The term “barb” refers to a widening or enlarged portion of the segment. In some implementations, the widening or enlarged portion may narrow down or taper to form the tip of the associated section. In some implementations, the widening or enlarged portion may not taper down to the tip, wherein the flexible line of the section continues below the barb before tapering to a tip. In the example illustrated, the tips of segment 2413 are pointed to better facilitate tissue penetration. In the examples, barbs 2418 are axially spaced from the tip of their respective end portions 2415 by no greater than 5 mm and in some implementations, no greater than 3 mm, where 3 mm is a desired distance for a desired depth of implantation.

In some implementations, the flexible line providing suture segment 2413 may comprise multiple spaced barbs. In some implementations, the flexible line providing suture segment 2413 may comprise a series of spaced barbs along its entire longitudinal length. For example, the flexible line may have a first plurality of barbs on a first side of and pointing away from a longitudinal center of the flexible line and a second plurality of barbs on a second side of and pointing away from the longitudinal center of the flexible line.

In some implementations, the flexible line forming suture segment 2413 has shape memory. In some implementations, the flexible line comprises portions that are elastic, wherein the suture segment 2413 may be stretched during penetration and insertion into the underlying tissue, wherein the suture segment will subsequently retract once in the tissue to draw together opposite edge portions of the cut or incision being sutured. In some implementations, the suture segment 2413 is formed from an organ absorbable and dissolvable material, such as Polydioxanone (PDS), eliminating the need to subsequently remove the suture. In some implementations, the suture segment 2413 has a length of at least 9 mm and no greater than 15 mm. In some implementations, the flexible line forming suture segment 34 has a diameter or thickness of at least 3-0 (United States Pharmacopeia (USP) numbering system for sutures, (0.25 mm) and no greater than 1 USP (0.4 mm).

Staple 2424 comprises a generally U-shaped member configured to engage the spaced end portions of suture segment 2413 and to be driven into tissue over and across a wound or cut by staple derived 2500. Staple 2424 comprises a center portion 2426 and prongs 2428 extending from the center portion 2426. Each of prongs 2428 comprises a substantially inflexible or rigid shaft (having a rigidity greater than that of the suture segment 2413. Each of prongs terminates at a terminal and that is pointed facilitate better tissue penetration. In the example illustrated, center portion 2426 comprises a lower face bottom slot or channel 2420 seven size are configured to at least partially receive those portions of flexible line of suture segment 2413 that extend between end portions 2415-1 and 2415-2. In other implementations, center portion 2426 may omit such a lower bottom facing channel 2427.

Each of prongs 2428 further comprises a barb catch 2450 configured to releasably engage or grip one of the barbs 2418 to resist separation of barbs 2418 when staple drive is impelling the prongs 2428 into tissue. At the same time, each of barbs 2428 is configured to release and separate from an engaged barb after the barb 2418 has been impelled into tissue and the staple 2424 is being withdrawn from the tissue. In some implementations, the shafts forming the two prongs 2428 are hollow, wherein each of barb catches 2450 comprises an upper closed edge of a side slot communicating with the hollow interior of the shaft. In some implementations, each of barb catches 2450 comprises a notch or hook formed in the shaft, above and axially spaced from the tip of the prong 2428, wherein the shafts forming each of prongs may be either solid or hollow. The two shafts forming each of prongs 2428 may be a single integrally formed shaft or distinct shafts joined to one another.

Staple drive 2500 comprises a mechanism configured to be coupled to the staple 2424 to impel the staple 2424 and the engaged the suture segment 34 into tissue 41 across the separated edges 42 of the tissue 41 being sutured (shown in FIG. 43). Staple drive 2500 is further configured to retract the staple 2424 from the tissue 41 while leaving the suture segment 2413 in the tissue 41. Staple drive 2500 may comprise a hydraulic, pneumatic or another mechanism operably coupled to center portion 2426 so as to exert force against center portion 2426 to impel tissue 41 with end portions 2415 and may likewise exert and opposite force against center portion 2426 so as to withdraw staple 2424 from the tissue. In some implementations, staple drive 2500 may utilize manual force to impel the staple 2424 and manual force to withdraw staple 2424. In some implementations, staple drive 2500 may utilize one or more springs for driving staple 2424 into tissue 41 and/or for withdrawing staple 2424 from tissue 41. The actuation of staple drive 2500 or the triggering of staple drive 2500 may be in response to a manual input to staple drive 2500.

FIGS. 44 and 45 illustrate an example prong 2628 for engaging one of the end portions of a suture segment. Prong 2628 may be utilized as one of the prongs of staple 2424 described above in system 2400. As should be appreciated, staple 2424 may include an additional prong similar to the prong 2628 shown in FIGS. 44 and 45.

Prong 2628 comprises a shaft 2629 that extends to a terminal end in the form of a pointed tip 2631. As shown in FIG. 45, the end of shaft 2629 has a tapered edge 2632 that extends downwardly or distally to the pointed tip 2631. Shaft 2629 has a hollow interior 2633 that extends to the tip 2631. In some implementations, the hollow interior terminates prior to reaching center portion 2426. In implementations where prong 2628 is part of staple 2424, and where center portion 2426 includes a downward facing bottom slot, the hollow interior may be continuous with the hollow interior of the downward facing bottom slot. In some implementations where prong 2628 is part of staple 2424 and where center portion 2426 includes a downward facing bottom slot, the hollow interior may terminate prior to reaching the hollow interior of the downward facing bottom slot.

Prong 2628 comprises a side slot 2640 that intersects the tapered edge 2632 and that extends upwardly away from tip 2631. Side slot 2640 has a sufficient width to accommodate the reception of a portion of a barb, such as barb 2418. Side slot 26 has a closed upper edge 2642 that forms an example barb catch 2650. Although the closed upper edge 2642 is illustrated as being concave and curved, the closed upper edge 2642 may alternatively have a polygon shape, such as a triangular shape, and rectangular shape or other polygon shapes.

FIG. 46 illustrates an example suture segment 2713 for being implanted using system 2400 with staple 2424 having prongs 2428, prongs 2628 or other prongs having barb catches. Suture segment 2713 comprises end portions 2714 (one of which is shown) which are formed by a flexible line 2715 having a terminal end in the form of a pointed tip 2716. Each of end portions 2714 of suture segment 2713 comprises barbs 2718-1, 2718-2 and 2718-3 (collectively referred to as barbs 2718). Barbs 2718 comprise hooks or other projections that extend outwardly from the central axis of flexible line 2715. In the example illustrated, the uppermost barb 2718-1 is configured to be at least partially received within side slot 2640 and to be captured or caught upon barb catch 2650. The remaining barbs 2718-2 and 2718-3 which extend between barb 2718-1 and pointed tip 2716 may have different lengths, different configurations and/or may be omitted. The additional barbs 2718-2 and 2718-3 provide enhanced securement or retention of the suture segment 2713 when implanted. In some implementations, barb 2718-1 may be omitted, wherein one of barbs 2718-2 or 2718-3 is likewise configured to be received within side slot 2640 and engaged by barb catch 2650.

FIG. 47 illustrates one of the end sections 2714 of suture segment 2713 engaged by one of prongs 2628 just prior to implantation of suture segment 2713 into the underlying tissue 41. As shown by FIG. 47, barb catch 2650 is releasably captured between an inside surface of barb 2718-1 and an outer surface of flexible line 2715. Portions of flexible line 2715 above barb 2718-1 continue upward along the exterior of prong 2628 towards center portion 2426 (shown in FIG. 43). In implementations where center portion 2426 has a lower facing bottom channel, those portions of flexible line 2715 extending between the two end portions of suture segment 2713 may be received within the lower facing bottom channel. In implementations where center portion 2426 lacks such a lower facing bottom channel, those portions of flexible line 2715 above barb 2718-1 may extend below the exterior of center portion 2426 to the other end section of suture segment 2713. In both cases, the shape of staple 2424 defines a corresponding U-shaped of the implanted suture segment 2713.

As further shown by FIG. 47, in some implementations, portions of flexible line 2715 and portions of the barb may be at least partially received within the hollow interior 2633 below closed end 2642 and along tapered edge 2632. Because the hollow interior 2633 of shaft 2629 below the barb engaged by barb catch 2650 at least partially receives prong 2718-3, and portions of flexible line 2715 below barb 2718-1, penetration into tissue 41 may better guided by prong 2628 and may be less traumatic. In other implementations, those portions of shaft 2629 below side slot 2640, along tapered edge 2632, may be filled or otherwise not hollow, wherein the hollow interior 2633 of prong 2628 terminates at a lower end of side slot 2640 at or slightly below the beginning of the tapering of edge 2632.

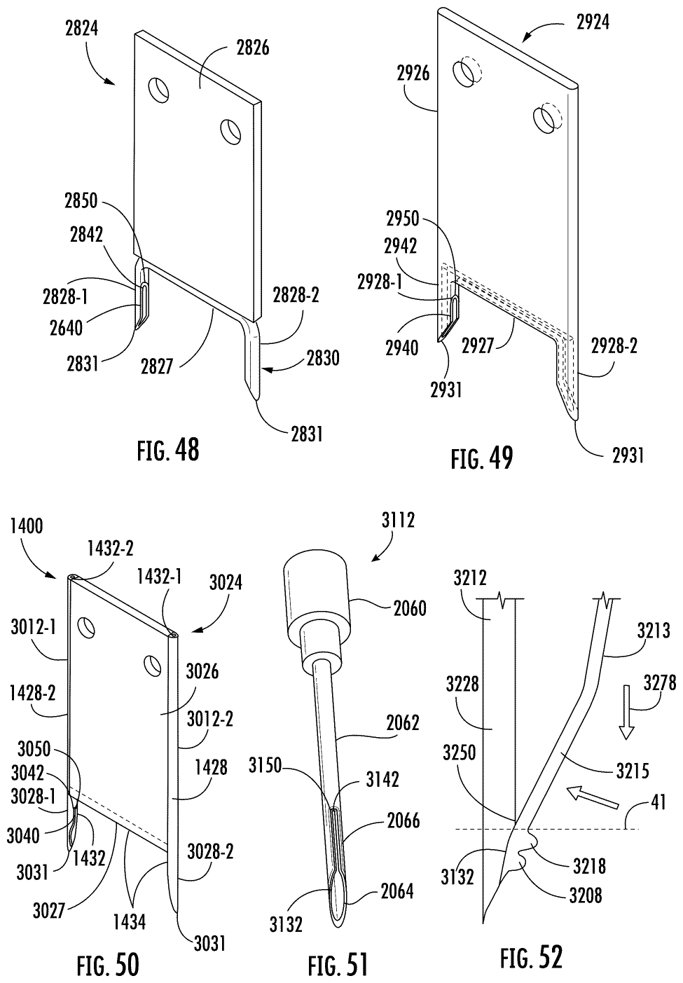

FIGS. 48, 49 and 50 illustrate alternative staples for system 2400. FIG. 48 illustrates an example staple 2824 which may be used in place of staple 2424 as part of system 2400. FIG. 48 illustrates an alternative staple 2824. As shown by FIG. 48, a hollow tube or hollow wire may have its end beveled to form tips 2831 and lower sides cut or opened to form side slots 2840 having closed upper ends 2842 that form barb catches 2850. The side slots 2840 may be configured to at least partially receive barbs 2848 or barbs 2718 described above.

In some implementations, central portions of the hollow tube or wire may also be cut or split to form a bottom facing slot 2827. In some implementations, the wire 28 comprise a solid (non-hollow) wire, wherein the axial ends are beveled to form the pointed tips 2831 and wherein a sitting saw is used to form the side slots 2940 which have the upper and 2842 serving as a barb catches. In some implementations, the slots may be formed by a slotting saw. Following such cutting, the hollow tube or wire 2830 may be bent to form prongs 2828-1 and 2828-2 (collectively referred to as prongs 2828), wherein such that the side slots 2840 face one another and such that the bottom slot 2827 extends downward. Thereafter, the bent wire, having a U-shaped profile, is affixed to a supporting plate 2826 by laser welding, adhesives or the like. In some implementations, the wire may be affixed to plate 2826, wherein the axial ends of wire 2830 are then bent, following such affixing to form the resulting prongs 2828.

FIG. 49 illustrates a staple 2924 which may be used in place of staple 2424 as part of system 2400. Staple 2924 comprises a plate 2926 having a lower end which is removed to form prongs 2928-1 and 2928-2 (collectively referred to as prongs 2928). Ends of the prongs 2928 are beveled to form pointed tips 2931 and portions of the prongs 2928 are slot to form side slots 2940 having closed upper ends 2942 that serve as barb catches 2950. Such side slots 2940 are sizing FIG. 2 at least partially receive barbs 2418 and/or barbs 2718 described above. In some implementations, regions of plate 2926 between prongs 2928 are further provided with an upwardly extending, downwardly facing slot 2927 which faces downward aside for the reception of central portions of the suture segment. The 2927 is sized to at least partially receive a central portion of a suture segment, such as suture segment 2413 or suture segment 2713.

FIG. 50 illustrates a staple 3024 which may be used in place of staple 2424 as part of system 2400. Staple 3020 comprises plate 3026 and pointed shafts or needles 3012-1 and 3012-2 (collectively referred to as needles 3012), the lower end portions of which form prongs 3028-1 and 3028-2 (collectively referred to as prongs 3028), respectively. As shown by Figure, upper end portions of each of needles 3012 are joined or affixed to side edges of plate 3026, such as by laser welding, adhesives or the like. The lower end portions of needles 3012 are each beveled to form pointed tips 3031. Interior sides of the lower ends of needles 3012 are cut, such as with a slotting saw, to form interior opposing side slots 3040 having closed upper edges 3042 which form barb catches 3050. Side slots 3040 are configured or dimensioned to at least partially receive barbs located proximate to opposite ends of a suture segment, such as barbs 2418 are barbs 2718 described above.

As shown by broken lines, in some implementations, a bottom facing lower slot 3027 may be cut into (such as with a slotting saw) (or molded as part of) the lower edge of plate 3026, wherein the lower slot 3027 is sized to at least partially receive central portions of a suture segment. In some implementations, the lower slot 3027 may be omitted. In some implementations, needles 3012 are hollow throughout, wherein the side slots communicate with the interior hollow regions of the needle and wherein the depth of the side slots is partially defined by the hollow interior of the needle. In other implementations, needles 3012, prior to slotting, may be solid (not hollow), wherein the depth of the side slots is based upon the depth of the cut forming the side slots. In some implementations, the side slotting of the lower ends of needles 3012 may occur prior to fixing of the needles to plate 3026. In other implementations, the side slotting of the lower ends of needles 3012 may be carried out after fixing of the needles to plate 3026. Because the staple shown in FIG. 48-50 comprise the described plates, such staples may be better suited for engagement by various forms of the staple drive 2500. For example, the staple shown in FIGS. 48-50 may be well-suited for use in the suture routing systems described in U.S. patent application Ser. No. 19/224,649, filed on May 30, 2025, by Thissen et al. and entitled SUTURING SYSTEM AND STAPLES, the full disclosure which is hereby incorporated by reference.

FIG. 51 illustrates an example needle 3112 for use in place of needle 2012 (described above) as part of the above described suturing systems that employee needle 2012. Needle 3112 is similar to needle 2012 except that the side slot 2066 has a closed upper edge 31 42 that forms a barb catch 3150. Those remaining components of needle 3112 which correspond to components of needle 2012 are numbered similarly. When mounted to a needle holder and forming a prong carriage, such as the prong carriage described above, needle 3112 extend opposite to another needle 3112 such that a side slots 2066 face one another. A suture segment loaded onto the prong carriage comprises barbs proximate to its opposite ends, wherein the barbs are received within side slots 2066 and are captured or engaged by barb catches 3150.

In some implementations, the axial length of side slot 2066 may be reduced from the length shown in FIG. 51. In some implementations, side slot 2066 may be omitted, where the angled or beveled edge 3132 of needle 3112 is sufficiently long to form a side opening having a sufficient depth to at least partially receive and capture a suture segment barb so as serve as a barb catch. FIG. 52 illustrates lower ends of an example needle 3212 which is similar to needle 3112 except that needle 3212 omits side slot 2066 and utilizes the upper closed edge of the beveled edge 3132 as the barb catch 3250. As shown by FIG. 52, a suture segment 3213 having barbs 3218 is engaged by those portions of needle 3212 forming prongs 3228, wherein portions of an upper most barb 3218 are received within an interior of prong 3228 defined by the beveled or angled edge 3132 and wherein the shoulder or notch between the upper most barb 3218 and the flexible line 3215 are contacted the upper edge of the beveled edge 3132 serving as barb catch 3250. As prong 3228 is driven downward in the direction indicated by arrow 3278, barb catch 3250 bears against the upper barb 3218 to drive suture segment 3213 into and below the surface of tissue 41 two implant the barbs 3218 below the tissue 41. Those portions of flexible line 3215 extending above the uppermost engage barb 3218 reside outside of prong 3228 during implantation.

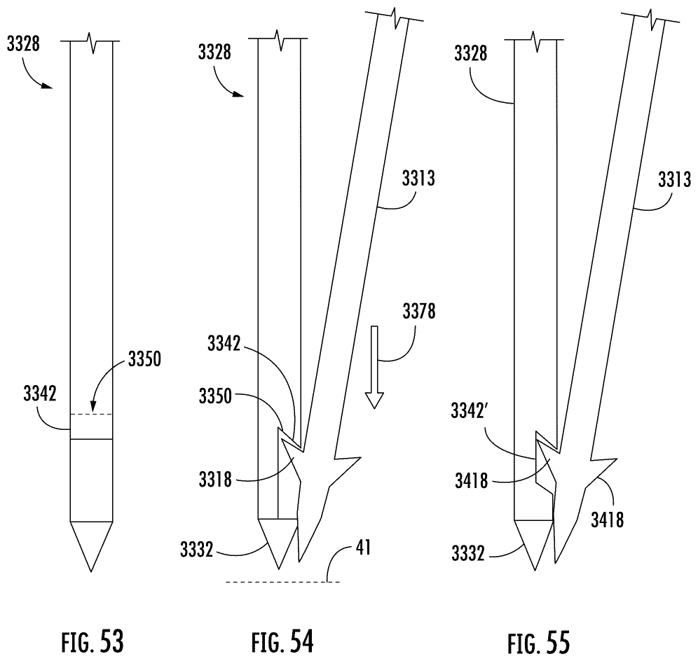

FIGS. 53 and 54 illustrate portions of an example (of a staple or of a needle). FIGS. 53 and 54 illustrate an example of a prong where the prong comprises an upwardly extending notch 3342 cut or otherwise formed an interior side of the prong 3328 and serving as the barb catch 3350. FIG. 54 illustrates end portions of a suture segment 3313 having at least one barb 3318 projecting into and received by the upward extending notch 3342 (which forms a hook) for engaging the barb 3318 to apply force and drive suture segment 3313 downward in a direction indicated by arrow 3378 during implanting of the suture segment 3313 below the surface of tissue 41. Suture seven 3313 comprise a center portion and another end portion likewise engaged by another prong during implementation. In the example illustrated, the notch 3342 extends to the beveled or pointed exterior 3332 of prong 3328, facilitating the reception of portions of suture segment 3313 below barb 3318 into the recess forming the notch. As shown by FIG. 55, in some implementations, prong 3328 may alternatively comprise a notch 3342′ that is shorter or that terminates prior to reaching the beveled outer surface 3332. In the implementation shown in FIG. 53-55, prong 3328 may be provided by a solid (not hollow) pointed shaft or needle having the notch or hook 3342, 3342′.

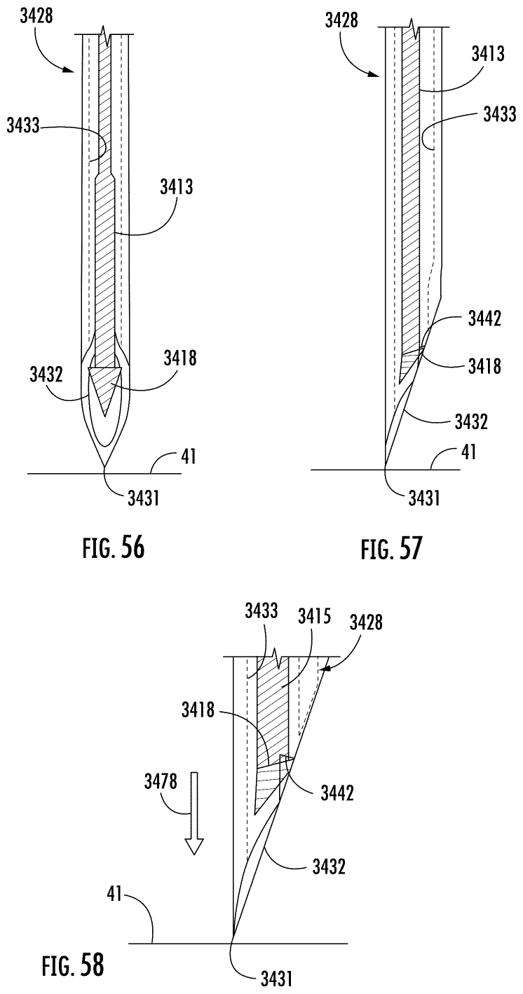

FIGS. 56, 57 and 58 illustrate portions of an example prong 3428 (of a staple or of a needle). FIGS. 56-58 illustrate an example of a prong where the prong at least partially receives those portions of the suture segment extending above (in a direction away from the pointed tip) the uppermost engaged or caught barb of the suture segment during implantation. Prong 3428 comprises a hollow interior 3433 which extends through the beveled or angled edge 3432 which terminates at a pointed tip 3431. As shown by FIG. 58, beveled edge 3332 comprise an upwardly extending notch 3442 configured to engage or capture a barb a suture segment. FIGS. 56-58 further illustrate end portions of an example suture segment 3413 having a barb 3418 caught by and received within notch 3442 as prong 3428 is being driven in the direction indicated by arrow 3478 towards and into tissue 41 by staple drive 2500. During such implantation, those portions of the flexible line 3415 extending above (away from tip 3431) barb 3418 are received within the hollow interior 3433 of prong 3428. Because a larger portion of the suture segment 3413 is contained within prong 3428, trauma to the tissue or the size of the opening formed by the implantation suture segment is reduced. In some implementations, the hollow interior 3433 may terminate prior to reaching the other prong 3428 that engages the barb of the opposite end of the suture segment 3413. In some implementations where the prong 3428 is part of a U-shaped staple, hollow interior 3433, configured to receive the flexible line of the suture segment 3413, may continually extend upwardly, across the horizontal portion of the staple and down the other prong of the staple.

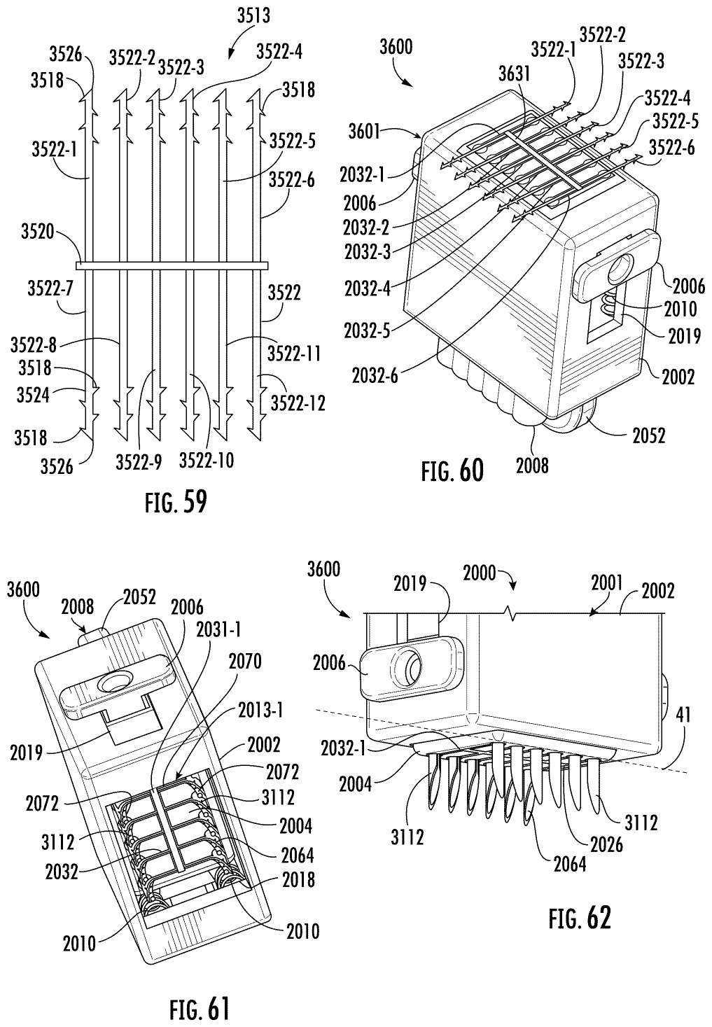

FIG. 59 illustrates an example one piece suture segment 3513 for use with a suturing system, such as a suturing system illustrated and described with respect to FIGS. 60, 61 and 62. Suture segment 3513 comprises a central length 3520 from which a plurality of legs 3522-1, 3522-2, 3522-3, 3522-4, 3522-5, 3522-6, 3522-7, 3522-8, 3522-9, 3522-10, 3522-11 and 3522-12 (collectively referred to as legs 3522) integrally extend. Legs 3522-1, 3522-2, 3522-3, 3522-4, 3522-5 and 3522-6 extend opposite to and along axes coincident with the axes of legs 35 22-7, 3522-relate, 3522-9, 3522-10, 3522-11 and 3522-12, respectively. At least portions of legs 3522 are formed from a flexible material. In some implementations, central length 3520, which serves as a spine for interconnecting each of legs 3522 is formed from the same material as that of legs 3522. In some implementations, central length 3520 may be formed from a material different than that of legs 3522. In some implementations, central length 3520 and legs 3522 are integrally formed as part of a single unitary body, such as by molding.

In some implementations, central length 3520 may be provided with an additional outer coating of the material which may result in central length 3520 having different properties, such as being stiffer, stronger and/or less flexible than the legs 3522. In some implementations, the diameter, size of central length 3520 may be different than that of legs 3522. For example, central length 3520 may be wider or thicker, whereas legs 3522 are thinner to facilitate greater flexibility or bending of legs 3522. In some implementations, legs 3522 may have a uniform diameter along their length (before the pointed tip and the outwardly extending barbs). In other implementations, portion of legs 3522 between barbs 3518 and central length 3520 may have a varying or nonuniform diameter, such as being narrower in particular regions to promote bending of the legs 3522 at particular locations along the axial lengths of legs 3522.

In some implementations, the flexible line forming the central length 3520 in each of legs 3522 of suture segment 3513 has shape memory. In some implementations, the flexible line comprises portions that are elastic, wherein the suture segment 3513 may be stretched during penetration and insertion into the underlying tissue, wherein the suture segment will subsequently retract once in the tissue to draw together opposite edge portions of the cut or incision being sutured. In some implementations, the suture segment 3513 is formed from an organ absorbable and dissolvable material, such as Polydioxanone (PDS), eliminating the need to subsequently remove the suture. In some implementations, the suture segment 3513 has a length of at least 9 mm and no greater than 15 mm. In some implementations, the flexible line forming suture segment 34 has a diameter or thickness of at least 3-0 (United States Pharmacopeia (USP) numbering system for sutures, (0.25 mm) and no greater than 1 USP (0.4 mm).

Each of legs 3522 forms an individual end portion 3524 of the suture segment 3513, each end portion 3524 being configured to be implanted into the tissue. Each end portion 3524 comprises a pointed tip 3526 and one or more barbs 3518. In some implementations, the lowermost barb 3518, the barb close to tip 3526, is no greater than 5 mm, and in some implementations, no greater than 3 mm from the tip 3526. In such implementations, the lowermost barb 3518 may be spaced from the tip 3526 by a non-zero distance. In some implementations, the tip 3526 may not be pointed. The barbs 3518 are configured to be driven into the underlying tissue by staple drive, not pulled into and through the underlying tissue. As result, the terminus or end of each of legs 3522 may be at or closely spaced to the distal or lowermost barb 3518, simplifying implantation.

FIGS. 60-61 illustrate an example suturing system 3600 having a suturing implanter 3601 for implanting suture segment 3513. Implanter 3601 is similar to implanter 2001 described above except that implanter 3601 comprises pressure foot 3604 and needles 3112 in place of pressure foot 2004 and needles 2012, respectively. Those remaining components of implanter 3601 which correspond to components of implanter 2001 are numbered similarly and function as described above.

Pressure foot 3604 is similar to pressure foot 2004 described above except that pressure foot 3604 comprises an additional channel 3631 in lower press face 2026. Channel 3631 is sized to receive central length 3520 of suture segment 3513, wherein the interior of channel 3631 is connected to and communicates with the interiors of each of channels 2032.

In some implementations, channel 3631 and/or channels 2032 may be omitted. For example, in some implementations, lower press face 2026 may alternatively comprise a resiliently compressible surface that partially deforms about a center portion of a suture segment or grips a suture segment. In some implementations, lower press face 2026 may be roughened to freshly contact and retain center portions of suture segments 2013. In some implementations, lower press face 2076 may be incompressible and/or smooth without any special features or material compositions to assist in retaining or gripping the center portion of a suture segment.

Needles 3112 are each similar to a needle 3112 described above with respect to FIG. 51. Each of needles 3112 are received within needle holder 2008 which form the prong carriage 2003 described above. As described above, each of directly opposing pair of needles 3112, opposite sides of channel 3631, comprises mutually facing or inwardly facing side slots 2066 which terminate at an upper edge 3142 which forms a barb catch 3150. The barb catch 3150 catches or engages a barb, typically the uppermost barb 3518, of an associated leg 3522 of suture segment 3513 during the concurrent implantation of the end portions 3524 of each of legs 3522 into tissue across a wound. The loading of suture segment 3513 into or onto implanter 3601, the implantation of suture segment 3513 by implanter 3601 and the withdrawal of implanter 3601 following the implantation of suture segment 3513 is similar to the above describe steps with respect to suture segments 2013 except that suture segment 3513 is a one piece suture segment with each of legs 3522 being interconnected by center set length 3520 provide greater control over the spacing and positioning of legs 3522 during and following implantation.

In some implementations, implanter 3601 may alternatively utilize needles 2012 described above in place of needles 3112. In some implementations, implanter 3601 may utilize needles having an portions that provide prongs similar to prongs 2628, 3328 or 3428 described above. In some implementations, needles 3112 may be utilized with other implanters, such as a staple having a number of prongs corresponding to the number of legs of suture segment 3513. In other implementations, suture segment 3513 may have a larger or fewer number of legs 3522 than that shown in FIG. 59. In some implementations, the provision of legs on opposite sides of central length 3520 may be staggered rather than opposite legs (for example, 3522-1 and 3522-7) extending along a single axis. In implementations where the legs are staggered, implanter 3601 may likewise comprise staggered positioning of the needles and staggered positioning of the channels 2032. In some implementations, suture segment 3513 may include more than one central length 3520 inter-joining or connecting legs 3522. In some implementations, each of the directly opposite pairs of coincident legs 3522 (such as legs 3522-1 and 3522-7) of suture segment 3513 may comprise a single line having barbs at opposite end, wherein the single line is fused, welded, bonded otherwise joined to central length 3520. Although central length 3520 is illustrated as continuously extending across or over each of legs 3522, in other implementations, central length 3520 may comprise individual segments extending between and connecting consecutive pairs of coincident legs 3522.

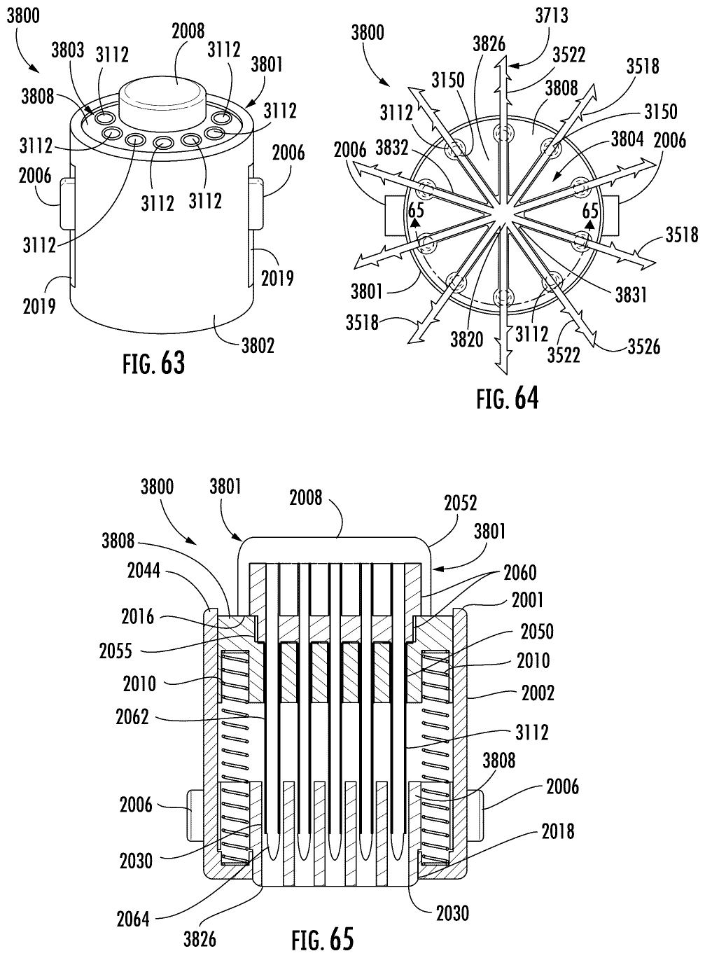

FIGS. 63, 64 and 65 illustrate an example suturing system 3800. System 3800 is configured to implant an example suture segment 3713, shown in FIG. 64. In addition to suture segment 3713, suturing system 3800 comprises suture implanter 3801. Implanter 3801 functions similarly to implanter 3601 except that implanter 3801 is configured to implant suture segment 3713. FIG. 64 illustrates the positioning of suture segment 3713 on an inverted implanter 3801 prior to retraction of foot 3804 to load implanter 3801 (as similarly described above with respect to implanter 2001 and the loading of suture segments 2013). FIG. 65 is a sectional view of implanter 3801 with the needles 3112 retracted, ready for loading with suture segment 3713.

As shown by FIG. 64, suture segment 3713 is similar to suture segment 3513 except that comprises a one piece suture segment having a hub 3820 from which the plurality of legs 3522 extend. Legs 3522 of suture segment 3713 are similar to legs 3522 of suture segment 3513. In the example illustrated, suture segment 3713 is illustrated as comprising 10 legs. In other implementations, the number of legs may vary. In the example illustrated, suture segment 3713 comprises five pairs of directly opposite coincident legs 3522 extending from hub 3820. In other implementations, suture segment 3713 may alternatively comprise legs that are staggered about hub 3820 so as to not be coincident or not be directly opposite to a corresponding leg. Because the legs 3522 of suture segment 3713 radially extend outwardly from hub 3820, suture segment 3713 is well-suited for forming an implanted suture that surrounds (by) 360° a wound, such as a puncture, drawing edges of the cutter wound together from all angles about the wound.

Although functioning similar to implanter 3601, to facilitate implantation of suture segment 3713, implanter 3801 has a generally cylindrical shape rather than a rectangular shape. Those components of implanter 3801 which correspond to components of implanter 3601, but for possibly their cylindrical or circular shape or layout, are illustrated in more detail with respect to implanter 3601, are described above and numbered similarly. Implanter 3801 comprises a cylindrical outer shell or housing 3802, a cylindrical pressure foot 3804, handles 2006, a cylindrical needle holder 3808, bias members 2010, and needles 3112. Needle holder 3808 and needles 3112 form a prong carriage 3803. In contrast to needle holder 2008, needle holder 3808 supports needles 3112 in a circular arrangement with the needle catches 3150 of the needles facing the center of the circle. In contrast to pressure foot 2004, pressure foot 3008 has lower press face 3826 having a center recess 3831 (in place of channel 3631) located and sized to receive hub 3820 and a series of radially extending channels 3832 having interiors that communicate with the interior of the center recess 3831, wherein recess 3831 and channels 3832 cooperate to receive portions of the loaded suture segment 3713.

Similar to the loading of onto implanter 3601, implanter 30 801 is loaded by positioning suture segment 3713 across the lower face as shown in FIG. 64. Thereafter, handles 206 are raised, lifting pressure foot 3804 upwards, relative to needle holder 3808, such that a lower ends of needles 3112 passed through and extend beyond the lower face of pressure foot 3804, wherein legs 3522 are also lifted upwards into housing 3802, causing the terminal ends of the legs 3522 to be bent at substantially 90° angles relative to those portions of legs 3522 that are closer to hub 3820. Such lifting continues until the uppermost bar 3518 of each leg 3522 catches upon the barb catch 3150 of the respective needles. At such point in time, suture segment 3713 is loaded, ready for implantation.

During implantation, the barb catches 3150 (shown in FIG. 51) catch or engage respective barbs, typically the uppermost barb 3518, of an associated leg 3522 of suture segment 3813, during the concurrent implantation of the end portions 3524 of each of legs 3522 into tissue across and encircling a wound. The implantation of suture segment 3713 by implanter 3801 and the withdrawal of implanter 3801 following the implantation of suture segment 3713 is similar to the above describe steps with respect to suture segments 2013 except that suture segment 3713 is a one piece suture segment with each of legs 3522 being interconnected by hub 3820 provide greater control over the spacing and positioning of legs 3522 during and following implantation. In particular, similar to suture segment implementation of the other systems described above, needle holder 3808 and pressure put 3804 are concurrently lowered to drive both the needles and the carried suture segment 3713 into the underlying tissue. Thereafter, the needle holder 3808 is a retracted, lifting the needles 3112 out of the underlying tissue while pressure foot 3804 remains in contact with the tissue, holding the suture segment 3713 in place as the needles 3112 are withdrawn. Following withdrawal of the needles, the pressure foot 3804 may be withdrawn from contact with the tissue.

Although suture segment 3713 is illustrated as having legs of a uniform length extending from hub 3820 such as the implanted barbs extend along a circular line or path about a wound, in other implementations, different legs of suture segment 3713 may have differing lengths extending from hub 3820. For example, the differing lengths may be such that the implanted barbs extend along an oval line or path about or along a wound. In such an implementation, implanter 3801 may alternatively support needles 3112 in an oval arrangement, rather than a circular arrangement. In such an implementation, suture segment 3713 may comprise legs 3522 of different lengths extending from hub 3820 such that the implanted barbs of the implanted suture segment extend about a wound a puncture in the shape of an oval rather than a circle. In such an implementation, the housing, pressure foot 3804 and needle holder would each be ovular cross-section and the recess 3831 and channels 3832 would likewise be relocated and reshaped to removably receive at least portions of the hub and legs of the alternative oval suture segment 3713.

As with implanter 3601, implanter 3801 may alternatively utilize needles 2012 described above in place of needles 3112. In some implementations, implanter 3801 may utilize needles having an portions that provide prongs similar to prongs 2628, 3328 or 3428 described above. In some implementations, needles 3112 may be utilized with other implanters, such as a staple having a number of prongs corresponding to the number of legs of suture segment 3713.

Although the present disclosure has been described with reference to example implementations, workers skilled in the art will recognize that changes may be made in form and detail without departing from the scope of the claimed subject matter. For example, although different example implementations may have been described as including features providing benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following claims is manifestly intended to be as broad as possible. For example, unless specifically otherwise noted, the claims reciting a single particular element also encompass a plurality of such particular elements. The terms “first”, “second”, “third” and so on in the claims merely distinguish different elements and, unless otherwise stated, are not to be specifically associated with a particular order or particular numbering of elements in the disclosure.

What is claimed is:

1. A suturing system comprising:
a suturing device for use with a suture segment having a first suture segment barb and a second suture segment barb, the suturing device comprising:
a first shaft terminating at a tip and having a first barb catch above the tip; and
a second shaft terminating at a second tip and having a second barb catch above the second tip.

2. The suturing system of claim 1, wherein the suturing device comprises:
a first side slot extending from the first suture segment barb catch in a direction toward the first tip, wherein the first suture segment barb catch comprises an upper end of the first side slot;
a second side slot facing the first side slot and extending from the second suture segment barb in a direction towards the second tip, wherein the first suture segment barb catch comprises an upper end of the first side slot.

3. The suturing system of claim 1, wherein the first barb catch comprises an upward extending first notch in the first shaft and wherein the second barb catch comprises an upwardly extending second notch in the second shaft.

4. The suturing system of claim 1, wherein the suturing device further comprises:
a needle holder; and
a first needle projecting from the needle holder and providing the first shaft; and
a second needle projecting from the needle holder and providing the second shaft.

5. The suturing system of claim 1, wherein the suturing device further comprises a third shaft spaced from the first shaft and the second shaft, the third shaft having a third suture segment barb catch.

6. The suturing system of claim 5, wherein the suturing device further comprises:
a needle holder; and
a first needle projecting from the needle holder and providing the first shaft; and
a second needle projecting from the needle holder and providing the second shaft.

7. The suturing system of claim 6 further comprising a pressure foot having a first passage through which the first shaft is movable and a second passage through which the second shaft is movable.

8. The suturing system of claim 7, wherein the pressure foot comprises a channel along a lower side and connecting the first passage and the second passage.

9. The suturing system of claim 5, wherein the suturing device comprises a staple comprising the first shaft, the second shaft, and an intermediate shaft extending between and interconnecting the first shaft and the second shaft.

10. The suturing system of claim 9, wherein the intermediate shaft is hollow and comprises a bottom slot.

11. The suturing system of claim 5, wherein the first shaft has a first side slot facing in a first direction and wherein the second shaft has a third side slot facing in a second direction oblique to the first direction.

12. The suturing system of claim 11, wherein the suturing device further comprises:
a needle holder; and
a first needle projecting from the needle holder and providing the first shaft; and
a second needle projecting from the needle holder and providing the second shaft.

13. The suturing system of claim 12 further comprising a pressure foot having a first passage through which the first shaft is movable and a second passage through which the second shaft is movable.

14. The suturing system of claim 13, wherein the pressure foot comprises a channel along a lower side and connecting the first passage and the second passage.

15. The suturing system of claim 11 further comprising the suture segment, the suture segment comprising:
a first end portion received in the first shaft and providing the first suture segment barb;
a second end portion received within the second shaft and providing the second suture segment barb; and
a third end portion received with the third shaft and providing a third suture segment barb.

16. The suturing system of claim 5, wherein the first shaft has a first side slot facing in a first direction and wherein the third shaft has a third side slot facing in a second direction parallel to the first direction.

17. The suturing system of claim 16 further comprising the suture segment, the suture segment comprising:
a first end portion received in the first shaft and providing the first suture segment barb;
a second end portion received within the second shaft and providing the second suture segment barb; and
a third end portion received with the third shaft and providing a third suture segment barb.

18. A one-piece suture segment comprising:
a first end portion having a first terminal end and a first barb;
a second end portion having a second terminal end and a second barb; and
a third end portion having a third terminal end and a third barb.

19. The one-piece suture segment of claim 18, wherein the first end portion extends parallel to the second end portion.

20. The one-piece suture segment of claim 18, wherein the first end portion extends along a first axis and wherein the second end portion extends along a second axis oblique to the first axis.
